(19) United States
(12) Patent Application Publication
(10) Pub. No.: US 2019/0214151 A1
(43) Pub. Date: Jul. 11, 2019

(54) SYSTEMS AND METHODS FOR GUIDING A PATIENT OR A CAREGIVER THEROF

(71) Applicant: PREEME+YOU, Oakland, CA (US)

(72) Inventors: Yayu Ren, Oakland, CA (US); Bree Landis Andrews, Chicago, IL (US)

(21) Appl. No.: 16/238,378

(22) Filed: Jan. 2, 2019

Related U.S. Application Data
(60) Provisional application No. 62/612,953, filed on Jan. 2, 2018.

(51) Int. Cl. G16H 80/00; A61B 5/00; A61B 5/0205; A61B 5/044

(52) U.S. Cl. CPC G16H 80/00; A61B 5/0015; A61B 5/0205; G16H 10/60; A61B 5/7435; A61B 5/7465; A61B 5/044

(57) ABSTRACT

Methods for guiding a patient or a caregiver thereof along a medical trajectory associated with a medical problem of the patient include: (a) displaying, by a processor, at least one designating query to the patient and/or the caregiver, the at least one designating query designed to ascertain a severity level of a first biomarker associated with the medical problem; (b) receiving a response from the patient and/or the caregiver to the at least one designating query; (c) correlating the response to the at least one designating query with a severity level of the first biomarker; and (d) displaying a designated severity level of the first biomarker in a graphical format. Systems for guiding a patient or a caregiver thereof along a medical trajectory associated with a medical problem of the patient are described.

### RELATED APPLICATIONS

This application claims the benefit of U.S. Provisional Application No. 62/612,953, filed Jan. 2, 2018. The entire contents of U.S. Provisional Application No. 62/612,953 are incorporated herein by reference, except that in the event of any inconsistent disclosure or definition from the present specification, the disclosure or definition herein shall be deemed to prevail.

### TECHNICAL FIELD

The present teachings relate generally to systems and methods for use in medicine and, in some embodiments, for use in neonatal medicine.

### BACKGROUND

Many medical problems, including but not limited to those requiring hospitalization, are characterized by a defined medical trajectory that begins with the inception of treatment and continues on at least until the time that the patient is deemed well enough to be discharged from the hospital. Along a typical medical trajectory, there are several milestones or other markers that a patient must meet. Satisfying these markers may be a prerequisite to discharge from the hospital. However, the pathway is not always linear, and it is not uncommon for a setback in the patient's condition to offset earlier improvements.

One significant problem in medicine today is that patients and their families often lack the medical knowledge and experience to comprehend in real time and in a visceral and substantive way the patient's precise medical status and/or position along a given medical trajectory. As a result, a perception may arise that the medical specialists engaged in a patient's treatment have preferential access to information about the patient's true medical status, which is being withheld from the patient and/or the patient's family members. This perception creates frustration and anxiety, and it is only exacerbated by the typically brief interactions that are common between doctors and their patients or family members. Indeed, the problem may be further exacerbated by the emotional vulnerability of the patients and their family members that commonly arises in hospital settings. The emotional toll of a medical problem and/or a hospital stay may result in a patient or family member's inability to think clearly and/or inability to articulate appropriate questions during the brief periods when the doctor or other medical specialist is accessible.

As difficult as it may be for an adult patient or family member thereof to comprehend in real time and in a visceral and substantive way the patient's precise medical status and/or position along a particular medical trajectory, the difficulty is even more acute in the case of children—and particularly premature infants. After an infant is born prematurely or is otherwise deemed to have serious medical issues, the infant may be placed in a neonatal intensive care unit (NICU) for treatment. The NICU is oftentimes an overwhelming experience for a parent and/or other caregiver, and the stress of the situation may result in barriers to a parent's engagement, knowledge, and comfort at their premature infant's beside.

### SUMMARY

By way of introduction, a first computer-implemented method for guiding a patient or a caregiver thereof along a medical trajectory associated with a medical problem of the patient in accordance with the present teachings includes: (a) displaying, by a processor, at least one designating query to the patient and/or the caregiver, the at least one designating query designed to ascertain a severity level of a first biomarker associated with the medical problem; (b) receiving, by the processor, a response from the patient and/or the caregiver to the at least one designating query; (c) correlating, by the processor, the response to the at least one designating query with a severity level of the first biomarker; and (d) displaying, by the processor, a designated severity level of the first biomarker in a graphical format.

A second computer-implemented method for guiding a patient or a caregiver thereof along a medical trajectory associated with a medical problem of the patient in accordance with the present teachings includes: (a) displaying, by a processor, at least one designating query to the patient and/or the caregiver, the at least one designating query being designed to ascertain a severity level of a first biomarker in a plurality of biomarkers associated with the medical problem, wherein each of the plurality of biomarkers has an assigned relative importance, and wherein severity levels of each of the plurality of biomarkers are ascertained sequentially in order of decreasing importance of the corresponding biomarker; (b) receiving, by the processor, a response from the patient and/or the caregiver to the at least one designating query; (c) correlating, by the processor, the response to the at least one designating query with a severity level of the first biomarker, wherein the first biomarker has primary importance among the plurality of biomarkers; (d) displaying, by the processor, at least one supportive query to the patient and/or the caregiver, the at least one supportive query designed to elicit information relevant to the first biomarker without influencing designation of the severity level thereof; (e) receiving, by the processor, a response from the patient and/or the caregiver to the at least one supportive query; (f) determining, by the processor, a first parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof; (g) correlating, by the processor, the determined first parameter and the designated severity level of the first biomarker with a bespoke designation description relevant to the first biomarker; (h) correlating, by the processor, the determined first parameter and the response to the at least one supportive query with bespoke supportive information relevant to the first biomarker; (i) displaying, by the processor, a designated severity level of the first biomarker in a graphical format; (j) displaying, by the processor, the bespoke designation description relevant to the first biomarker in a graphical format; and (k) displaying, by the processor, the bespoke supportive information relevant to the first biomarker in a graphical format.

A computer-implemented method for guiding a caregiver of a premature infant along a medical trajectory associated with the infant's organ maturation in accordance with the present teachings includes: (a) displaying, by a processor, at least one designating query to the caregiver, the at least one designating query designed to ascertain a severity level of a breathing biomarker; (b) receiving, by the processor, a response from the caregiver to the at least one designating query; (c) correlating, by the processor, the response to the at least one designating query with a severity level of the breathing biomarker, wherein the breathing biomarker is assigned primary importance among a plurality of biomarkers associated with organ maturation of the infant; (d) displaying, by the processor, at least one supportive query to the caregiver, the at least one supportive query designed to elicit information relevant to the breathing biomarker without influencing designation of the severity level thereof; (e) receiving, by the processor, a response from the caregiver to the at least one supportive query; (f) displaying, by the processor, at least one additional designating query to the caregiver, the at least one additional designating query designed to ascertain a severity level of at least one additional biomarker associated with the organ maturation, the at least one additional biomarker selected from the group consisting of eating, temperature control, sleeping, growth, and a combination thereof; (g) receiving, by the processor, a response from the caregiver to the at least one additional designating query; (h) correlating, by the processor, the response to the at least one additional designating query with a severity level of the at least one additional biomarker; (i) displaying, by the processor, at least one additional supportive query to the caregiver, the at least one additional supportive query designed to elicit information relevant to the at least one additional biomarker without influencing a designation of the severity level thereof; (j) receiving, by the processor, a response from the caregiver to the at least one additional supportive query; (k) determining, by the processor, a parameter selected from the group consisting of (i) a duration of time for which the designated severity level of the breathing biomarker has been assigned to the patient, (ii) a locale of the infant's medical treatment, (iii) a number of times that the infant has previously been assigned the designated severity level of the breathing biomarker, (iv) a duration of time for which the designated severity level of the at least one additional biomarker has been assigned to the infant, (v) a number of times that the infant has previously been assigned the designated severity level of the at least one additional biomarker, and (vi) a combination thereof; (l) correlating, by the processor, the determined parameter and the designated severity level of the breathing biomarker with a bespoke designation description relevant to the breathing biomarker; (m) correlating, by the processor, the determined parameter and the designated severity level of the at least one additional biomarker with a bespoke designation description relevant to the at least one additional biomarker; (n) correlating, by the processor, the determined parameter and the response to the at least one supportive query with bespoke supportive information relevant to the breathing biomarker; (o) correlating, by the processor, the determined parameter and the response to the at least one additional supportive query with bespoke supportive information relevant to the at least one additional biomarker; (p) displaying, by the processor, a designated severity level of the breathing biomarker in a graphical format; (q) displaying, by the processor, a designated severity level of the at least one additional biomarker in a graphical format; (r) displaying, by the processor, the bespoke designation description relevant to the breathing biomarker in a graphical format; (s) displaying, by the processor, the bespoke supportive information relevant to the breathing biomarker in a graphical format; (t) displaying, by the processor, the bespoke designation description relevant to the at least one additional biomarker in a graphical format; and (u) displaying, by the processor, the bespoke supportive information relevant to the at least one additional biomarker in a graphical format.

A first system for guiding a patient or a caregiver thereof along a medical trajectory associated with a medical problem of the patient in accordance with the present teachings includes a processor coupled to a non-transitory memory. The processor is operative to execute computer program instructions to cause the processor to: (a) display at least one designating query to the patient and/or the caregiver, the at least one designating query designed to ascertain a severity level of a first biomarker associated with the medical problem; (b) receive a response from the patient and/or the caregiver to the at least one designating query; (c) correlate the response to the at least one designating query with a severity level of the first biomarker; and (d) display a designated severity level of the first biomarker in a graphical format.

A second system for guiding a patient or a caregiver thereof along a medical trajectory associated with a medical problem of the patient in accordance with the present teachings includes a processor and a non-transitory memory coupled with the processor. The system further includes: (a) first logic stored in the non-transitory memory and executable by the processor to cause the processor to display at least one designating query to the patient and/or the caregiver, the at least one designating query designed to ascertain a severity level of a first biomarker associated with the medical problem; (b) second logic stored in the non-transitory memory and executable by the processor to cause the processor to receive a response from the patient and/or the caregiver to the at least one designating query; (c) third logic stored in the non-transitory memory and executable by the processor to cause the processor to correlate the response to the at least one designating query with a severity level of the first biomarker; and (d) fourth logic stored in the non-transitory memory and executable by the processor to cause the processor to display a designated severity level of the first biomarker in a graphical format.

A third system for guiding a patient or a caregiver thereof along a medical trajectory associated with a medical problem of the patient in accordance with the present teachings includes a processor and a non-transitory memory coupled with the processor. The system further includes: (a) first logic stored in the non-transitory memory and executable by the processor to cause the processor to display at least one designating query to the patient and/or the caregiver, the at least one designating query being designed to ascertain a severity level of a first biomarker in a plurality of biomarkers associated with the medical problem, wherein each of the plurality of biomarkers has an assigned relative importance, and wherein severity levels of each of the plurality of biomarkers are ascertained sequentially in order of decreasing importance of the corresponding biomarker; (b) second logic stored in the non-transitory memory and executable by the processor to cause the processor to receive a response from the patient and/or the caregiver to the at least one designating query; (c) third logic stored in the non-transitory memory and executable by the processor to cause the processor to correlate the response to the at least one designating query with a severity level of the first biomarker, wherein the first biomarker has primary importance among the plurality of biomarkers; (d) fourth logic stored in the non-transitory memory and executable by the processor to cause the processor to display at least one supportive query to the patient and/or the caregiver, the at least one supportive query designed to elicit information relevant to the first biomarker without influencing designation of the severity level thereof; (e) fifth logic stored in the non-transitory memory and executable by the processor to cause the processor to receive a response from the patient and/or the caregiver to the at least one supportive query; (f) sixth logic stored in the non-transitory memory and executable by the processor to cause the processor to determine a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof; (g) seventh logic stored in the non-transitory memory and executable by the processor to cause the processor to correlate the determined parameter and the designated severity level of the first biomarker with a bespoke designation description relevant to the first biomarker; (h) eighth logic stored in the non-transitory memory and executable by the processor to cause the processor to correlate the determined parameter and the response to the at least one supportive query with bespoke supportive information relevant to the first biomarker; (i) ninth logic stored in the non-transitory memory and executable by the processor to cause the processor to display a designated severity level of the first biomarker in a graphical format; (j) tenth logic stored in the non-transitory memory and executable by the processor to cause the processor to display the bespoke designation description relevant to the first biomarker in a graphical format; and (k) eleventh logic stored in the non-transitory memory and executable by the processor to cause the processor to display the bespoke supportive information relevant to the first biomarker in a graphical format.

A system for guiding a caregiver of a premature infant along a medical trajectory associated with the infant's organ maturation in accordance with the present teachings includes a processor and a non-transitory memory coupled with the processor. The system further includes (a) first logic stored in the non-transitory memory and executable by the processor to cause the processor to display at least one designating query to the caregiver, the at least one designating query being designed to ascertain a severity level of a breathing biomarker; (b) second logic stored in the non-transitory memory and executable by the processor to cause the processor to receive a response from the caregiver to the at least one designating query; (c) third logic stored in the non-transitory memory and executable by the processor to cause the processor to correlate the response to the at least one designating query with a severity level of the breathing biomarker, wherein the breathing biomarker is assigned primary importance among a plurality of biomarkers associated with organ maturation of the infant; (d) fourth logic stored in the non-transitory memory and executable by the processor to cause the processor to display at least one supportive query to the caregiver, the at least one supportive query designed to elicit information relevant to the breathing biomarker without influencing designation of the severity level thereof; (e) fifth logic stored in the non-transitory memory and executable by the processor to cause the processor to receive a response from the caregiver to the at least one supportive query; (f) sixth logic stored in the non-transitory memory and executable by the processor to cause the processor to display at least one additional designating query to the caregiver, the at least one additional designating query designed to ascertain a severity level of at least one additional biomarker associated with the organ maturation, the at least one additional biomarker selected from the group consisting of eating, temperature control, sleeping, growth, and a combination thereof; (g) seventh logic stored in the non-transitory memory and executable by the processor to cause the processor to receive a response from the caregiver to the at least one additional designating query; (h) eighth logic stored in the non-transitory memory and executable by the processor to cause the processor to correlate the response to the at least one additional designating query with a severity level of the at least one additional biomarker; (i) ninth logic stored in the non-transitory memory and executable by the processor to cause the processor to display at least one additional supportive query to the caregiver, the at least one additional supportive query designed to elicit information relevant to the at least one additional biomarker without influencing a designation of the severity level thereof; (j) tenth logic stored in the non-transitory memory and executable by the processor to cause the processor to receive a response from the caregiver to the at least one additional supportive query; (k) eleventh logic stored in the non-transitory memory and executable by the processor to cause the processor to determine a parameter selected from the group consisting of (i) a duration of time for which the designated severity level of the breathing biomarker has been assigned to the patient, (ii) a locale of the infant's medical treatment, (iii) a number of times that the infant has previously been assigned the designated severity level of the breathing biomarker, (iv) a duration of time for which the designated severity level of the at least one additional biomarker has been assigned to the infant, (v) a number of times that the infant has previously been assigned the designated severity level of the at least one additional biomarker, and (vi) a combination thereof; (l) twelfth logic stored in the non-transitory memory and executable by the processor to cause the processor to correlate the determined parameter and the designated severity level of the breathing biomarker with a bespoke designation description relevant to the breathing biomarker; (m) thirteenth logic stored in the non-transitory memory and executable by the processor to cause the processor to correlate the determined parameter and the designated severity level of the at least one additional biomarker with a bespoke designation description relevant to the at least one additional biomarker; (n) fourteenth logic stored in the non-transitory memory and executable by the processor to cause the processor to correlate the determined parameter and the response to the at least one supportive query with bespoke supportive information relevant to the breathing biomarker; (o) fifteenth logic stored in the non-transitory memory and executable by the processor to cause the processor to correlate the determined parameter and the response to the at least one additional supportive query with bespoke supportive information relevant to the at least one additional biomarker; (p) sixteenth logic stored in the non-transitory memory and executable by the processor to cause the processor to display a designated severity level of the breathing biomarker in a graphical format; (q) seventeenth logic stored in the non-transitory memory and executable by the processor to cause the processor to display a designated severity level of the at least one additional biomarker in a graphical format; (r) eighteenth logic stored in the non-transitory memory and executable by the processor to cause the processor to display the bespoke designation description relevant to the breathing biomarker in a graphical format; (s) nineteenth logic stored in the non-transitory memory and executable by the processor to cause the processor to display the bespoke supportive information relevant to the breathing biomarker in a graphical format; (t) twentieth logic stored in the non-transitory memory and executable by the processor to cause the processor to displaying the bespoke designation description relevant to the at least one additional biomarker in a graphical format; and (u) twenty-first logic stored in the non-transitory memory and executable by the processor to cause the processor to display the bespoke supportive information relevant to the at least one additional biomarker in a graphical format.

A non-transitory computer readable storage medium in accordance with the present teachings has stored therein data representing instructions executable by a programmed processor for guiding a patient or a caregiver thereof along a medical trajectory associated with a medical problem of the patient. The storage medium includes instructions for: (a) displaying at least one designating query to the patient and/or the caregiver, the at least one designating query designed to ascertain a severity level of a first biomarker associated with the medical problem; (b) receiving a response from the patient and/or the caregiver to the at least one designating query; (c) correlating the response to the at least one designating query with a severity level of the first biomarker; and (d) displaying a designated severity level of the first biomarker in a graphical format.

### BRIEF DESCRIPTION OF THE DRAWINGS

FIG. 1 shows a representative example of a graphical display screen soliciting entry of basic information from a parent.

FIG. 2 shows a representative example of a graphical display screen showing representative examples of questions displayed to a parent.

FIG. 3 shows a representative example of a graphical display screen with a circular graphical interface showing five biomarkers on a single day.

FIG. 4 shows a representative example of a graphical display screen showing a circular graphical interface of one biomarker over five days.

FIG. 5 shows a representative example of a graphical display screen showing a pentagonal representation of five biomarkers.

FIG. 6 shows a representative example of a graphical display screen showing a pentagonal representation of five biomarkers on a single day.

FIG. 7 shows a representative example of exemplary instructions for using a system in accordance with the present teachings.

FIG. 8 shows a flowchart of a representative first process for guiding a patient or a caregiver thereof along a medical trajectory associated with a medical problem of the patient in accordance with the present teachings.

FIG. 9 shows a flowchart of a representative second process for guiding a patient or a caregiver thereof along a medical trajectory associated with a medical problem of the patient in accordance with the present teachings.

FIG. 10 shows a block diagram of a representative system for guiding a patient or a caregiver thereof along a medical trajectory associated with a medical problem of the patient in accordance with the present teachings.

FIG. 11 shows a representative general computer system for use with a system in accordance with the present teachings.

FIGS. 12A through 12G show representative examples of a series of graphical display screens associated with designating the severity level of a breathing biomarker.

FIG. 13 shows a flowchart of a representative first process (“Forced Designation”) for designating the severity level of a biomarker in accordance with the present teachings.

FIG. 14 shows a flowchart of a representative second process (“Ask All”) for designating the severity level of a biomarker in accordance with the present teachings.

FIG. 15 shows a flowchart of a representative third process (“Tiebreaker”) for designating the severity level of a biomarker in accordance with the present teachings.

FIG. 16 shows a flowchart of a representative fourth process (“Question Set”) for designating the severity level of a biomarker in accordance with the present teachings.

### DETAILED DESCRIPTION

Systems and methods for guiding a patient and/or a caregiver thereof along a medical trajectory associated with a medical problem of the patient have been discovered and are described herein. The systems and methods described herein are applicable to all manner of medical problems having a defined medical trajectory characterized by one or more biomarkers—including but not limited to a trajectory that begins with the inception of a patient's treatment and continues up to some predetermined metric by which the desired improvement in the patient's health is determined (e.g., the day that the patient is deemed well enough to be discharged from a hospital). In accordance with the present teachings, as further explained below, the one or more biomarkers may be assessed for severity level and used to chart the patient's progress along a medical trajectory.

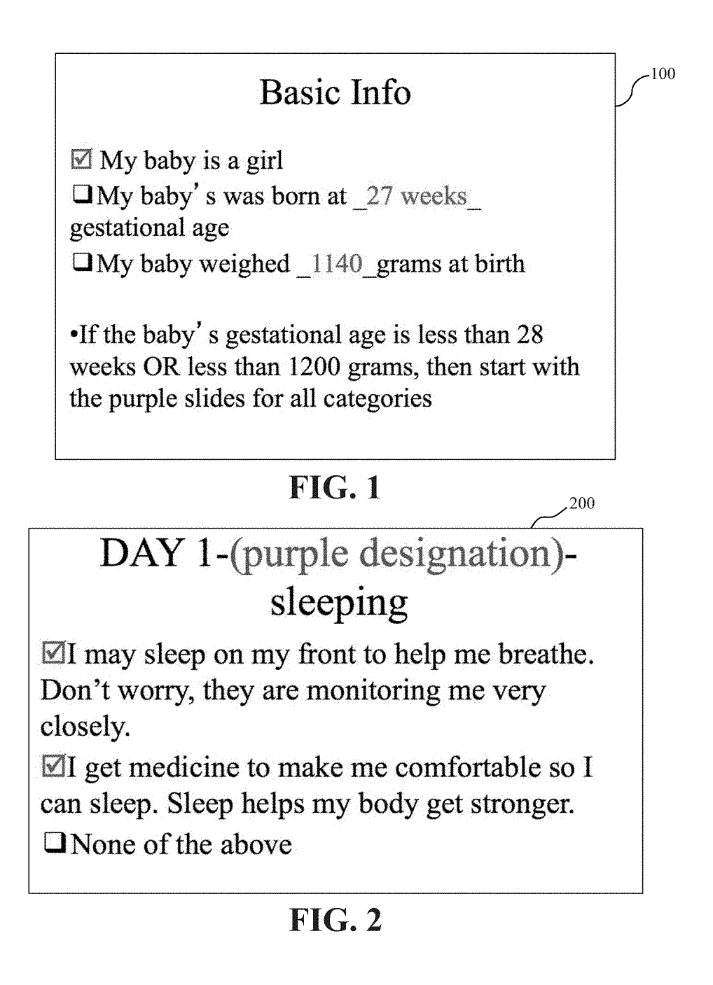

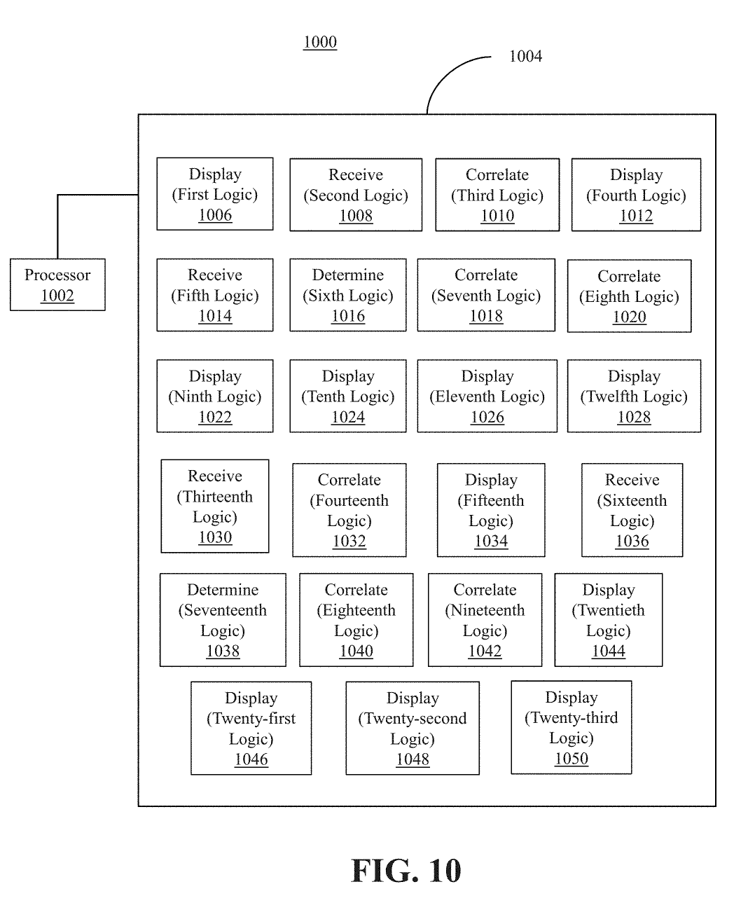

The systems and methods described herein may be applicable in human medicine as well as in veterinary medicine. In some embodiments, systems and methods in accordance with the present teachings are used in the practice of human medicine and, in some embodiments, for the treatment of premature infant patients (e.g., preemies). Representative medical problems for which systems and methods in accordance with the present teachings may be used include but are not limited to premature birth, cancer, heart disease, stroke, infection, diabetes, influenza, pneumonia, joint replacement, organ transplant, all manner of surgeries, and/or the like, and combinations thereof. As a representative and non-limiting example of a system and method in accordance with the present teachings, an exemplary application in the field of neonatal medicine for preemie patients having a medical trajectory associated with organ maturation is described below in reference to FIGS. 1 through 7.

A system and method for human infant medical pathways and organ maturation trait analysis in accordance with the present teachings includes a system for determining the staging of a premature infant's medical pathway and organ system maturation for survival outside the uterus during neonatal intensive care treatment and after discharge. In some embodiments, five general biomarkers are used: breathing, eating, temperature control, sleeping, and growth. In accordance with the present teachings, a graphic may be displayed for each of the five biomarkers based on levels of severity and dependence on neonatal intensive care technologies and medicine for survival. In some embodiments, the graphic is in color. In a representative and non-limiting example, the colors may be selected as follows: purple for severe; blue for moderate; orange for mild; and yellow for minimal/none. The display of biomarkers graphics may show a single biomarker over several days, or all biomarkers on one day. In some embodiments, other graphics may be used to represent the system processor's analysis of the biomarkers.

In accordance with the present teachings, traits such as clinical, physiological, behavioral markers, assessments, indicators, medications, and/or prognoses in varying combinations may be analyzed to provide a more complete picture of an individual neonatal infant or groups of neonatal infants.

Systems and methods in accordance with the present teachings for guiding a caregiver of a premature infant along a medical trajectory associated with the infant's medical problem is based at least in part upon one or more or the following fundamental discoveries: (a) subjects under the medical care provided in neonatal intensive care units (NICUs) typically need to overcome complicated medical pathways that require extensive medical knowledge, effort, and time to communicate; (b) infant organ maturation level is complicated to ascertain and communicate; (c) analyzing medical pathways and organ maturation of infants under NICU care helps to ensure their survival and increase their chances for a positive developmental outcome after hospital discharge; (d) deconstructing complicated clinical inputs and medical processes for infants under NICU care improves understanding of medical conditions and best practices; (e) communicating the analysis in a graphic form that deconstructs complicated and varied clinical-medical inputs and organ system biomarkers improves quality of NICU care; (f) communicating the complexity of a NICU infant's medical condition in systematized, easy-to-understand, color-coded language and graphics empowers parents and other caregivers; (g) communicating the complexity of a NICU infant's medical condition in real time improves the hospital experience for parents (and other caregivers); (h) communicating the complexity of a NICU infant's medical condition in a systemized, self-guided manner decreases parental stress, increases empowerment at the bedside, and improves understanding of the infant's medical condition; and/or (i) the system as a whole may improve infant mortality, morbidity, and/or overall health.

In accordance with the present teachings, a framework is provided for taking complicated and varied clinical and medical inputs through a series of questions, analyzing the inputs, and displaying the results of the analysis as a graphic. The questions may be self-guided and/or utilized as a communication guide to (a) empower parents, family members, and other caregivers to improve their comprehension of medical knowledge and the medical condition of the infant, (b) decrease parental anxiety and stress, and/or (c) improve parental engagement with the infant and medical staff and experts. In accordance with the present teachings, parental social-emotional experience may be aligned with the severity levels of the medical pathway and organ maturation of NICU infants

In accordance with the present teachings, curated and timely neonatal health information and parental self-care information for improved neonatal outcomes is provided in real time. Communication between parents and medical staff, medical caretakers, nurses, and doctors (collectively “medical staff”) is thus facilitated. The systems and methods in accordance with the present teachings may aid communication between medical staff and parents in order to quickly bring all participants onto the same page.

In some embodiments, systems and methods are provided that track the infant's medical condition daily, or as often as parents and/or medical staff would like, and that allow parents and/or medical staff to track the medical pathway and level of organ maturation health of the infant over time and in real time.

In some embodiments, parents and/or medical staff will first determine basic information about the premature infant, which may include weeks of gestation, weight at birth, and/or other information deemed relevant by medical staff. This information is inputted into a framework. In some embodiments, the framework includes a system processor configured to analyze the inputted information and, based on the information, to display appropriate questions to the parent in different health areas referred to as biomarkers. The parent will answer questions about each biomarker based on the daily medical and clinical inputs of the infant. Each time answers are input, the system processor analyzes the answers according to a tested framework that stages the severity of the premature infant's medical condition and level of organ systems maturation. The system is configured to analyze the input answers and determine a health level for the infant based on the input answers and medical knowledge framework. In some embodiments, the system is further configured to translate the information received for each biomarker into a color-coded graphic.

Systems and methods in accordance with the present teachings provide a framework in which to track the health of an infant and to provide realistic expectations for the parent. In addition, systems and methods in accordance with the present teachings may empower parents to engage with medical knowledge and/or plan for their infant's present and future care. Furthermore, systems and methods in accordance with the present teachings may provide social-emotional support mechanism for parents. In accordance with the present teachings, the tool is designed to give parents a way to participate in the health care being provided to their baby, while simultaneously educating them about realistic outcomes for infants in the NICU. The tool increases engagement of parents with their NICU preemie, which in turn may lead to better prepared parents, potentially earlier NICU discharge, healthier babies with better outcomes, and better communication between medical staff and families. Better parental preparation and earlier discharge from the NICU may be beneficial for many reasons including but not limited to an estimated cost-savings of $5,000 per day or more, as well as future savings vis-à-vis developmental outcome care, hospital readmission, and/or further medical care costs stemming from lack of parental confidence in caring for their infants following hospital discharge.

In some embodiments, the tool may help sync an infant's needs with the parent's response, social-emotional state, and confidence level. In a full-term baby, by the middle of the interaction, a mother's “peaks” and the baby's “peaks” are much more in sync. By contrast, in a preterm baby, the parent and baby are often out-of-sync. By using a tool in accordance with the present teachings, parents may be educated with respect to their infant's maturation and needs, which in turn may lead to more in-sync interactions between parent and baby, improved developmental outcomes, improved parent-infant relationships, and/or decreased parental anxiety.

In some embodiments, there are five main categories or biomarkers that a system in accordance with the present teachings analyzes to establish medical pathways and organ maturation in the infant. These five biomarkers include: (1) breathing (including the assessment of all related organ systems and physiologies required for breathing); (2) eating (including the assessment of all related organ systems and physiologies required for eating); (3) temperature control (including the assessment of all related organ systems and physiologies required for temperature control); (4) sleeping (including the assessment of all related organ systems and physiologies required for sleeping); and (5) growth (including the assessment of all related organ systems and physiologies required for growth). The above-described fiver biomarkers are meant to be representative rather than limiting, and it is to be understood that one or more of these biomarkers may be eliminated and/or one or more additional biomarkers may be added, as will be readily appreciated by one of ordinary skill in the art.

In some embodiments, a system for guiding a caregiver of a premature infant along a medical trajectory associated with the infant's organ maturation in accordance with the present teachings displays one or a plurality of questions to a parent. The questions may be displayed on a screen—in some embodiments, a touch screen—of a computer, tablet, mobile device, or other interactive internet-connected device (hereafter collectively “mobile device”).

In some embodiments, the parent may respond by entering information into the mobile device. This action empowers parents in the care of their preemie. If the parent does not know the answer to a particular question, the display screen may prompt the parent to ask medical staff to provide the missing information to assist the parent to answer the question. In some embodiments, the system may ask the parent for biomarker information each day or at other time intervals.

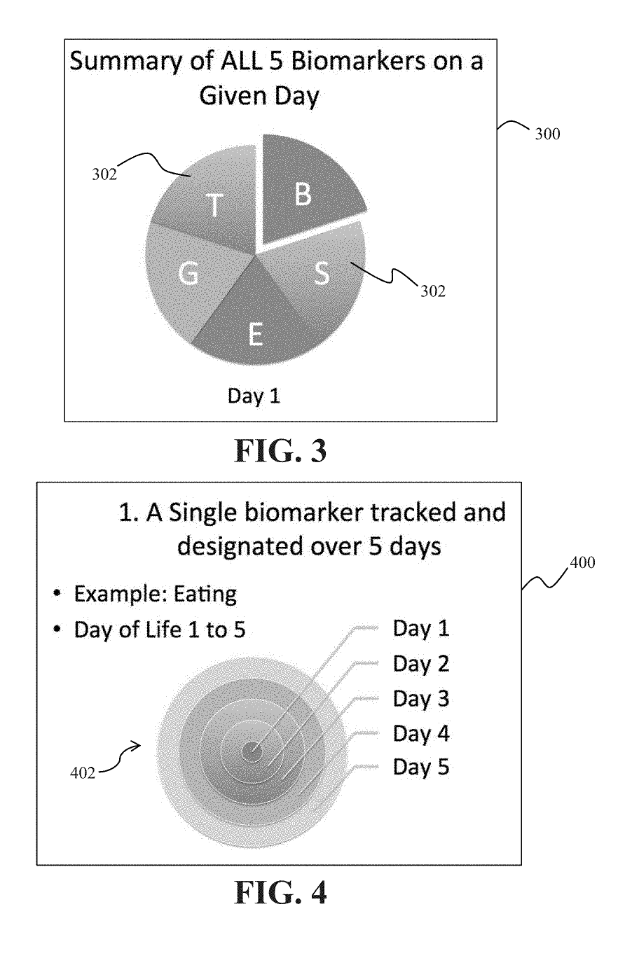

FIG. 1 shows a representative example of a graphical display screen 100 soliciting entry of basic information from a parent. As shown in FIG. 1, the display screen 100 asks for basic information about the preemie. Basic information may include but is not limited to gestational age, weight at birth, sex, name, and any other information. FIG. 2 shows a representative example of a graphical display screen 200 showing representative examples of questions displayed to a parent. The parent may answer the questions by entering responses into the mobile device. The mobile device transmits the received information to the system processor.

After receiving the parental responses, the system processor analyzes the basic information to evaluate the health level for the infant. After analysis, the system processor may transmit to the parent's mobile device a display that includes particular questions that are appropriate for the health level of the infant for each biomarker category. The particular questions may be displayed on one screen or on a series of screens.

In some embodiments, the system processor assigns a graphic to each of the biomarkers based on levels of severity and dependence on neonatal intensive care technologies and medicine for survival. In some embodiments, the graphic is color-coded with purple signifying severe, blue signifying moderate, orange signifying mild, and yellow signifying minimal/none.

As a representative and non-limiting example, if the infant was born at less than 27 weeks gestational age, the system may display questions in the purple category, where a purple graphical interface represents the lowest level of health for a preemie.

NICU infants progress at different rates and may be at different health levels for each of the respective biomarkers. Therefore, their medical pathways and organ maturation levels change over time. Based on the information entered by the parent regarding the infant's health, a system in accordance with the present teachings may display questions that are relevant to the appropriate health level for each biomarker. In some embodiments, the system will analyze the information provided by the parent and will display the appropriate questions for each biomarker and each health level thereof.

In some embodiments, the system processor receives the answers input by the parents on all health categories once a day or several times a day and processes that information. After analyzing the parental responses and analyzing the medical information contained in the system processor database, the system may display a graphical interface that corresponds to the health status of the preemie. In some embodiments, the graphic includes a color, wherein each color is associated with a particular health level.

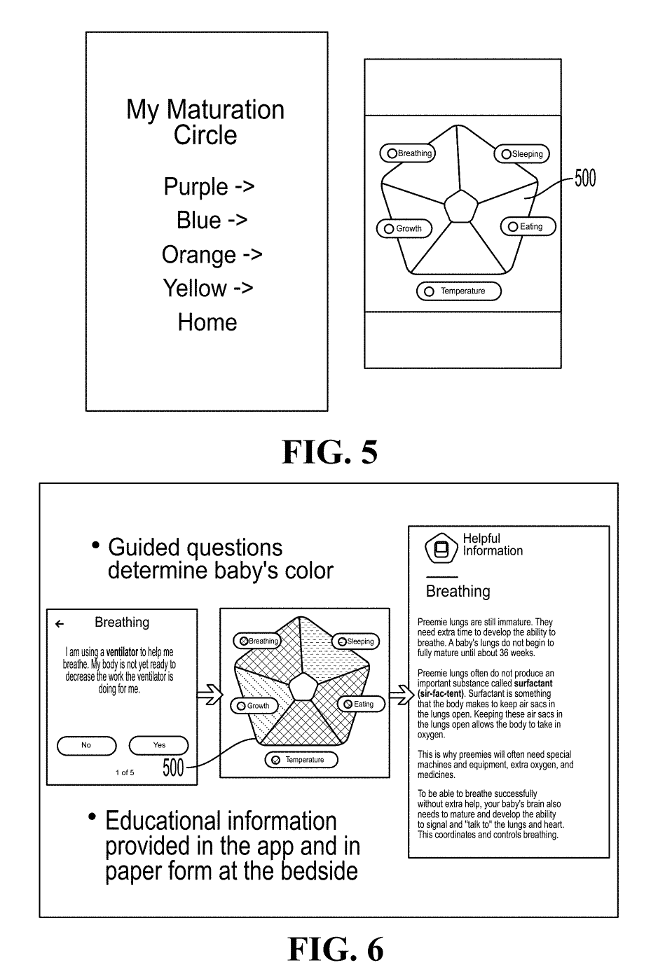

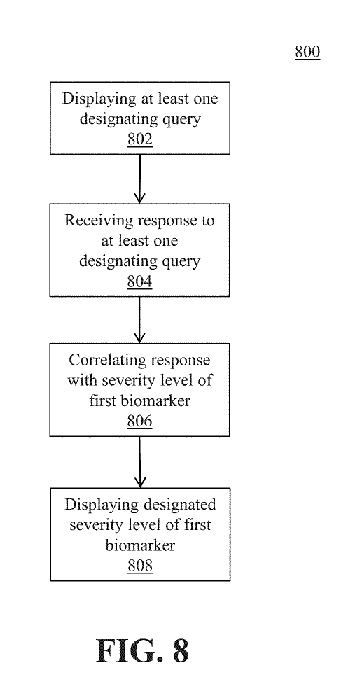

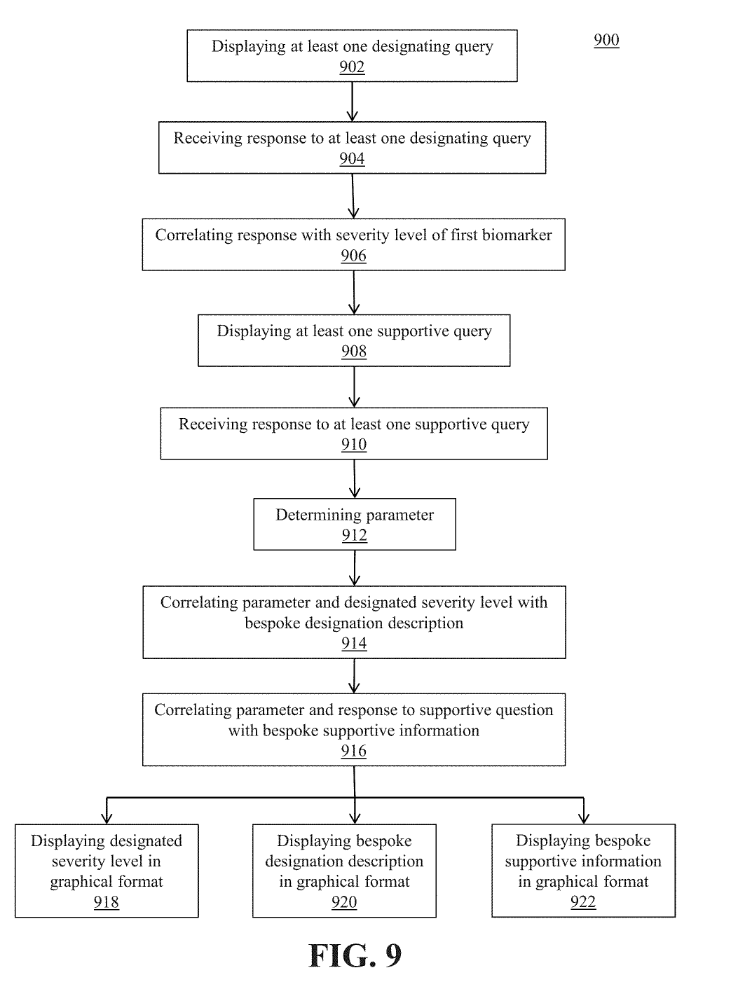

In some embodiments, the colors for each biomarker are put into another graphical interface for each day. FIG. 3 shows a representative example of a graphical display screen 300 with a circular graphical interface showing five biomarkers on a single day. In some embodiments, as shown in FIG. 3, the graphical interface 300 may include a pie-chart circle, with each “pie” slice 302 representing a separate biomarker. In other embodiments, as shown in FIGS. 5 and 6, the graphical interface may include a pentagon 500. It is to be understood that all manner of other shapes may alternatively be used and that the circular shape shown in FIG. 3 and the pentagonal shape shown in FIGS. 5 and 6 are merely illustrative.

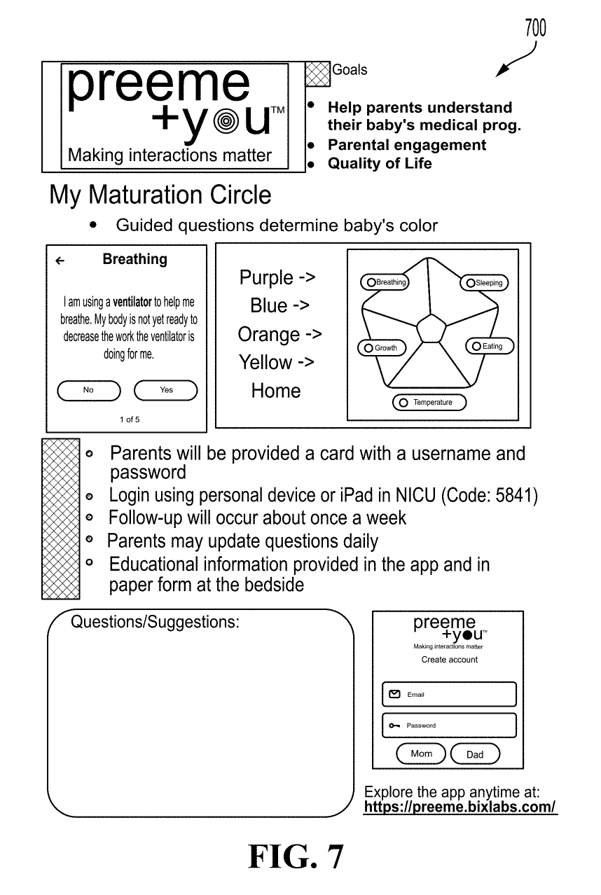

In some embodiments, a tool in accordance with the present teachings may also provide a display that shows the preemie's progress for a single biomarker over time. For example, the system may analyze the responses each time a parent enters information into the system. After synthesizing, analyzing, and processing this information, and evaluating the information based on logical steps in synthesizing and analyzing the medical information in the system database, the system may assign a graphic for each biomarker. This graphic may be transmitted to and displayed on the parent's mobile device. In some embodiments, as shown in FIG. 4, the system may display a graphic 400 regarding each particular health biomarker each day over several days based on the parent's answers and subsequent to analysis. This information may be summarized in a concentric color-coded circle graphic 402. In other embodiments, the graphical interface may display the health biomarker over hours or over other time units. In such a way, parents may visualize their preemie's progress over time. There are many permutations and clinical perspectives that may be useful to parents, caretakers, and/or the like. In some embodiments, as shown in FIG. 4, the graphic may include concentric color-coded circles 402, wherein each circle represents one day. In other embodiments, the graphic may have a pentagonal or other shape.

In some embodiments, in addition to providing a graphical interface of the preemie's status over time, a tool in accordance with the present teachings may also provide information to parents that relates to the various health categories. Benefits that may arise from using a system or method for guiding a caregiver of a premature infant along a medical trajectory associated with the infant's medical problem in accordance with the present teachings include but are not limited to one or more of the following: (a) decreased parental stress during infant's NICU care; (b) increased parental engagement during infant's NICU care; (c) provision of a self-guided framework for parents to understand the medical condition of their infant without the physical presence of a medical expert; (d) provision of an intuitive alignment between the infant's medical pathway and the parent's NICU experience; (e) provision of a logical and powerful approach that clarifies many complicated clinical inputs regarding the infant's medical condition; (f) provision of a comprehensive color graphics design showing infant's medical condition from different perspectives; (g) provision of a logical and powerful way to understand, analyze, and synthesize an individual's or a group of infants' medical condition; (h) provision of a logical and powerful way to understand, analyze, and synthesize individual biomarker inputs or combinations of biomarker inputs by severity from individual or combinations of biomarker inputs (e.g., overall, daily, weekly, by severity, by single or different combinations of biomarkers, and/or the like, and combinations thereof); (i) provision of a tool that facilitates the tracking of the infant's medical condition in real time (e.g., captures changes in real time); (j) provision of a tool that readily allows medical experts to categorize infants' medical conditions for improved research on best medical outcomes and medical practices; (k) provision of a new color wheel language to understand the infant's medical condition that reduces communication time; (l) provision of a system that helps different medical specialties and teams connect and communicate well together; (m) provision of a tool that enables medical experts to develop new strategies to improve health outcomes for infants under NICU care; (n) provision of a tool that enables medical experts to better track and communicate best neonatal medical practices; (o) provision to parents of a self-guided and informative bedside ritual; (p) provision of a tool to allow clinicians and medical experts to verify that parents accurately understand their infant's medical condition; and/or (q) provision of a tool for standardized communication alignment between medical caretakers, clinicians, parents, and others involved in the care of NICU infants.

It is to be understood that elements and features of the various representative embodiments described below may be combined in different ways to produce new embodiments that likewise fall within the scope of the present teachings.

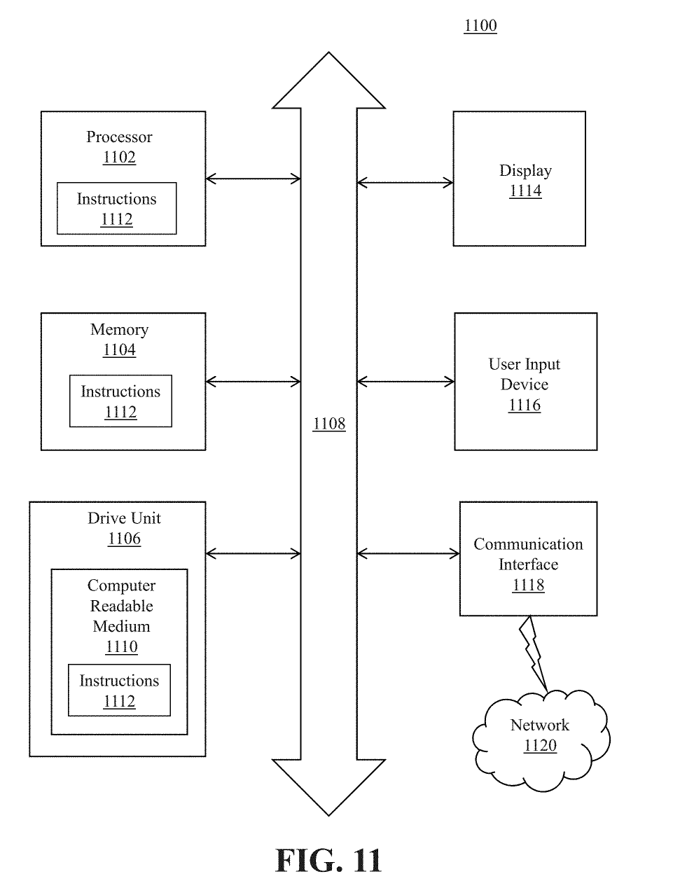

By way of general introduction, as shown in FIG. 8, an exemplary method 800 for guiding a patient or a caregiver thereof along a medical trajectory associated with a medical problem of the patient in accordance with the present teachings includes: (a) displaying 802 at least one designating query to the patient and/or the caregiver, the at least one designating query designed to ascertain a severity level of a first biomarker associated with the medical problem; (b) receiving 804 a response from the patient and/or the caregiver to the at least one designating query; (c) correlating 806 the response to the at least one designating query with a severity level of the first biomarker; and (d) displaying 808 a designated severity level of the first biomarker in a graphical format.

As used herein, the term “patient” refers to any living entity in need of medical treatment and encompasses both humans (e.g., adults, children, seniors, infants, preemies, etc.) and animals (e.g., dogs, cats, horses, pigs, sheep, birds, reptiles, fish, etc.).

As used herein, the term “caregiver” refers broadly to any individual concerned with the treatment and/or medical wellbeing of a patient. Representative caregivers in accordance with the present teachings include but are not limited to parents, grandparents, siblings, other family members of the patient, pet owners, doctors, nurses, residents, therapists, technicians, medical assistants, and/or the like, and combinations thereof.

The term “query” refers to any request for information that is answerable in either a binary (e.g., “yes” or “no”; “1” or “2”; etc.) or a non-binary (e.g., check all boxes that apply; etc.) fashion. As used herein, the term “query” is intended to include requests for information posed in the form of a question (e.g., “Is your baby on a ventilator?”) as well as declarative statements that are to be either confirmed or denied (e.g., “Your baby is on a ventilator”).

In some embodiments, a method for guiding a patient or a caregiver thereof along a medical trajectory associated with a medical problem of the patient in accordance with the present teachings is implemented using a computer and, in some embodiments, one or a plurality of the acts of (a) displaying 802, (b) receiving 804, (c) correlating 806, and/or (d) displaying 808 described above are performed by one or a plurality of processors. The processors are able to render more quickly and consistently than a person. For time constrained medical environments, processor-based image generation assists diagnosis and/or treatment in ways that a human created image could not. In some embodiments, one or more of the one or the plurality of processors include graphics processing units (GPUs). In other embodiments, one or more of the one or the plurality of processors include central processing units (CPUs). In some embodiments, methods in accordance with the present teachings are implemented entirely on GPUs. In some embodiments, GPUs provide improved and/or faster performance.

In some embodiments, the first biomarker is only one of a plurality of biomarkers associated with the medical problem. In other embodiments, the first biomarker is the only biomarker associated with the medical problem. For embodiments in which the first biomarker is only one of a plurality of biomarkers associated with the medical problem, the method 800 shown in FIG. 8 may further include (e) displaying at least one additional designating query to the patient and/or the caregiver, the at least one additional designating query designed to ascertain a severity level of at least one additional biomarker associated with the medical problem; (f) receiving a response from the patient and/or the caregiver to the at least one additional designating query; (g) correlating the response to the at least one additional designating query with a severity level of the at least one additional biomarker; and (h) displaying a designated severity level of the at least one additional biomarker in a graphical format. In this way, the severity level of each of the plurality of biomarkers may be determined.

For embodiments in which the first biomarker is only one of a plurality of biomarkers associated with the medical problem, each of the plurality of biomarkers may, in some embodiments, be assigned a relative importance, and the severity levels of the individual biomarkers may be determined sequentially in order of decreasing importance. For example, if a breathing biomarker is assigned primary importance amongst a plurality of biomarkers, the first severity level to be ascertained will be that of the breathing biomarker.

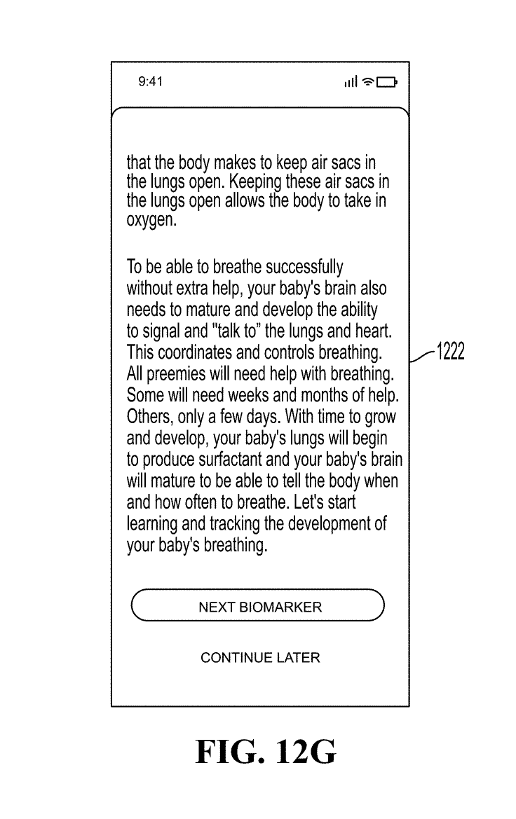

FIGS. 12A through 12G show representative examples of a series of graphical mobile device display screens associated with designating the severity level of a breathing biomarker in accordance with the present teachings. It is to be understood that the representative depictions shown in FIGS. 12A through 12G are meant to be purely illustrative and non-limiting. FIG. 12A shows a home screen 1200 in which each of five representative biomarkers for a preemie—breathing, growth, temperature control, eating, and sleeping—are represented by icons. The breathing biomarker is shown at icon 1202, the growth biomarker is shown at icon 1204, the temperature control biomarker is shown at icon 1206, the eating icon is shown at icon 1208, and the sleeping icon is shown at icon 1210.

In some embodiments, the breathing biomarker is assigned primary importance, such that the first severity level to be ascertained will be that of the breathing biomarker. Thus, as shown in FIG. 12A, engagement of the start button 1209 on the screen 1200 results in display of a designating query 1210 designed to ascertain the severity level of the breathing biomarker, as shown in FIG. 12B. The designating query 1210 “I am using a ventilator to help me breathe” is displayed on the screen 1200 and the user is prompted to enter either “yes” 1212 or “no” 1214, as further shown in FIG. 12B. If the answer to the designating query 1210 is affirmative, as shown in FIG. 12C, the designated severity level of the breathing biomarker is conclusively determined to be at the most serious level (e.g., purple).

Once a designated severity level has been assigned to a biomarker, a designation description 1222 relevant to that biomarker may be provided, as shown in FIGS. 12F and 12G. In some embodiments, the designation description provides information (e.g., text) of a type that a doctor might otherwise attempt to convey orally to the patient and/or caregiver during a face-to-face meeting. This information may include generic background information about the severity level of the biomarker, a preview of therapeutic options that may be implemented, tests that may be ordered, and/or the like. In some embodiments, the designation description may be tailored for consumption by an audience without medical training (e.g., a parent and/or the like). For embodiments in which a designation description relevant to a particular biomarker is to be provided, the method 800 shown in FIG. 8 may further include (e) correlating the designated severity level of the first biomarker with a designation description relevant to the first biomarker; and (f) displaying the designation description relevant to the first biomarker in a graphical format. For embodiments in which the first biomarker is only one of a plurality of biomarkers associated with the medical problem, separate designation descriptions may be provided for each of the biomarkers once their designated severity levels have been ascertained.

In some embodiments, the designation description provides information (e.g., in a textual format) that may be individualized or bespoke for a specific patient based on one or more variable parameters including but not limited to how long the patient has been assigned the designated severity level of the biomarker. For example, the designation description for a premature infant whose breathing biomarker has been purple for only one day may differ from the designation description for a premature infant whose breathing biomarker has been purple for twenty-three days. Other variable parameters that may be used to tailor a bespoke designation description in accordance with the present teachings include but are not limited to the locale or culture in which the treatment is being administered, the preferences of the specific medical staff involved in the patient's treatment, whether the designated severity level is indicative of an improvement in the patient's condition or whether it is the result of a backslide, and/or the like, and combinations thereof. For embodiments in which a bespoke designation description relevant to a particular biomarker is provided, the method 800 shown in FIG. 8 may further include (e) determining a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof; (f) correlating the determined parameter with a bespoke designation description relevant to the first biomarker; and (g) displaying the bespoke designation description relevant to the first biomarker in a graphical format.

In some embodiments, a single designating query is used to determine the severity level for a biomarker. For example, an affirmative response to the single query 1210 in FIGS. 12C and 12D (“I am using a ventilator to help me breathe”) may be probative in determining that the breathing biomarker should be assigned the highest severity level. However, in other embodiments, multiple designation queries may be posed as part of the process of ascertaining the severity level for a given biomarker. In some embodiments, multiple affirmative response may be needed in order to conclusively determine that a biomarker should be designated as having a certain severity level.

In some embodiments, additional queries that do not influence the designation of a severity level, but which nonetheless are designed to collect information relevant to a particular biomarker, may also be posed. These additional queries are meant to be supportive in nature and may be posed regardless of whether only one or a plurality of designation queries is needed to designate the severity level of a biomarker.

Once a designated severity level has been assigned to a biomarker, a supportive query designed to elicit further information relevant to that biomarker may be posed as shown in FIGS. 12C and 12D. For example, as shown in FIG. 12C, a representative supportive query that may be posed in connection with the breathing biomarker is the supportive query 1216 shown in FIGS. 12C and 12D (“I am using IV sedation to keep my body calm and give it as much rest as possible”). A further example of a representative supportive query shown in FIG. 12D is the supportive query 1218 (“My team wants to see an X ray of how I'm doing almost every day or every other day”). Although a caregiver's response to the supportive queries 1216 and 1218 will not affect the designation of the severity level for the breathing biomarker, these supportive queries are designed to reflect what the patient and/or caregiver may already be observing at the patient's bedside (e.g., the infant is connected to an IV and undergoes frequent x-ray diagnostic tests), thereby mitigating the caregiver's anxiety and stress, and instilling a basic understanding of what is happening and why.

In some embodiments, supportive information relevant to the corresponding biomarker may be provided in view of the patient or caregiver's response to a supporting query. This supportive information may include generic explanatory statements (e.g., the reason why frequent x-ray tests are being ordered) and/or generally supportive comments intended to reduce fear in the patient or caregiver (e.g., “these tests are merely routine and do not cause any pain”). In some embodiments, the supportive information may be incorporated into the designation description (e.g., the designation description 1222 shown in FIGS. 12F and 12G). In other embodiments, the supportive information may be displayed separately. In some embodiments, the supportive information may be tailored for consumption by an audience without medical training (e.g., a parent and/or the like).

For embodiments in which one or more supportive queries not influencing the designated severity level of a biomarker are to be presented, the method 800 shown in FIG. 8 may further include (e) displaying at least one supportive query to the patient and/or the caregiver, the at least one supportive query designed to elicit information relevant to the first biomarker without influencing designation of the severity level thereof; (f) receiving a response from the patient and/or the caregiver to the at least one supportive query; (g) correlating the response to the at least one supportive query with supportive information relevant to the first biomarker; and (h) displaying the supportive information relevant to the first biomarker in a graphical format. For embodiments in which the first biomarker is only one of a plurality of biomarkers associated with the medical problem, separate supportive information may be provided for each of the biomarkers once their designated severity levels have been ascertained.

In some embodiments, the supportive information may be individualized or bespoke for a specific patient based on one or more variable parameters including but not limited to the practice norms in the specific medical unit involved in the patient's treatment. For example, the supportive query for a premature infant whose breathing biomarker is purple and who is being treated in a unit that favors twice-daily x-ray diagnostic tests may differ from the supportive query for a premature infant being treated in a unit that limits x-ray diagnostic testing to once every two days. Other variable parameters that may be used to tailor bespoke supportive information in accordance with the present teachings include but are not limited to the amount of time that the patient has been assigned the designated severity level for a particular biomarker, the locale or culture in which the treatment is being administered, the preferences of the specific medical staff involved in the patient's treatment, whether the designated severity level is indicative of an improvement in the patient's condition or whether it is the result of a backslide, and/or the like, and combinations thereof. For embodiments in which a bespoke supportive information relevant to a particular biomarker is provided, the method 800 shown in FIG. 8 may further include (e) displaying at least one supportive query to the patient and/or the caregiver, the at least one supportive query designed to elicit information relevant to the first biomarker without influencing designation of the severity level thereof; (f) receiving a response from the patient and/or the caregiver to the at least one supportive query; (g) determining a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof; (h) correlating the determined parameter and the response to the at least one supportive query with bespoke supportive information relevant to the first biomarker; and (i) displaying the bespoke supportive information relevant to the first biomarker in a graphical format.

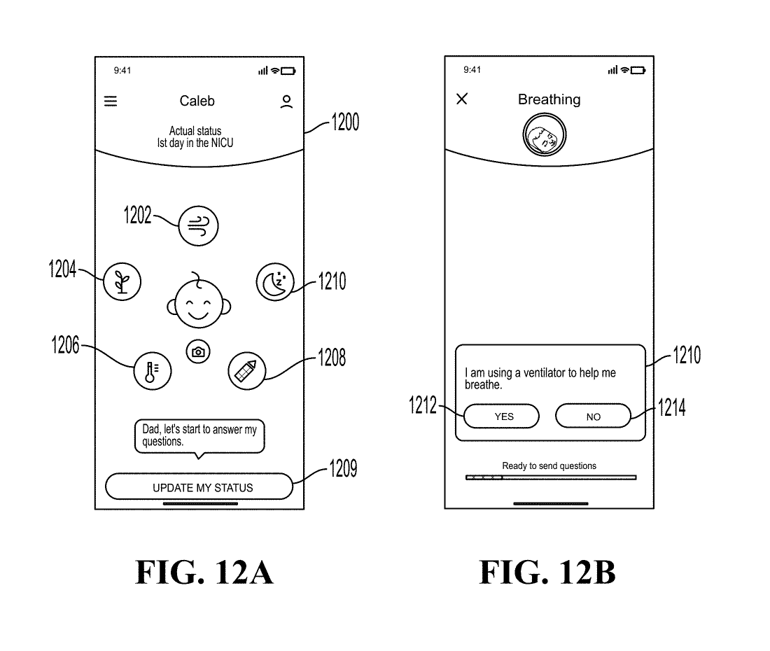

As noted above, elements and features of the various representative embodiments described herein may be combined in different ways to produce new embodiments that likewise fall within the scope of the present teachings. By way of example, as shown in FIG. 9, an exemplary method 900 for guiding a patient or a caregiver thereof along a medical trajectory associated with a medical problem of the patient in accordance with the present teachings includes: (a) displaying 902 at least one designating query to the patient and/or the caregiver, the at least one designating query being designed to ascertain a severity level of a first biomarker in a plurality of biomarkers associated with the medical problem, wherein each of the plurality of biomarkers has an assigned relative importance, and wherein severity levels of each of the plurality of biomarkers are ascertained sequentially in order of decreasing importance of the corresponding biomarker; (b) receiving 904 a response from the patient and/or the caregiver to the at least one designating query; (c) correlating 906 the response to the at least one designating query with a severity level of the first biomarker, wherein the first biomarker has primary importance among the plurality of biomarkers; (d) displaying 908 at least one supportive query to the patient and/or the caregiver, the at least one supportive query designed to elicit information relevant to the first biomarker without influencing designation of the severity level thereof; (e) receiving 910 a response from the patient and/or the caregiver to the at least one supportive query; (f) determining 912 a first parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof; (g) correlating 914 the determined first parameter and the designated severity level of the first biomarker with a bespoke designation description relevant to the first biomarker; (h) correlating 916 the determined first parameter and the response to the at least one supportive query with bespoke supportive information relevant to the first biomarker; (i) displaying 918 a designated severity level of the first biomarker in a graphical format; (j) displaying 920 the bespoke designation description relevant to the first biomarker in a graphical format; and (k) displaying 922 the bespoke supportive information relevant to the first biomarker in a graphical format.

In some embodiments, a method for guiding a patient or a caregiver thereof along a medical trajectory associated with a medical problem of the patient in accordance with the present teachings is implemented using a computer and, in some embodiments, one or a plurality of the acts of (a) displaying 902, (b) receiving 904, (c) correlating 906, (d) displaying 908, (e) receiving 910, (f) determining 912, (g) correlating 914, (h) correlating 916, (i) displaying 918, (j) displaying 920, and/or (k) displaying 922 described above are performed by one or a plurality of processors. The processors are able to render more quickly and consistently than a person. For time constrained medical environments, processor-based image generation assists diagnosis and/or treatment in ways that a human created image could not. In some embodiments, one or more of the one or the plurality of processors include graphics processing units (GPUs). In other embodiments, one or more of the one or the plurality of processors include central processing units (CPUs). In some embodiments, methods in accordance with the present teachings are implemented entirely on GPUs. In some embodiments, GPUs provide improved and/or faster performance.

It is to be understood that the relative ordering of some acts shown in the flowcharts of FIGS. 8 and 9 are meant to be merely representative rather than limiting, and that alternative sequences may be followed. Moreover, it is likewise to be understood that additional, different, or fewer acts may be provided, and that two or more of these acts may occur sequentially, substantially contemporaneously, and/or in alternative orders. By way of a non-limiting and representative example, in FIG. 9, the act 908 of displaying at least one supportive query is shown as preceding (a) the act 918 of displaying the designated severity level in graphical format, (b) the act 920 of displaying a bespoke designation description in graphical format, and (c) the act 922 of displaying bespoke supportive information in a graphical format. However, in alternative embodiments, these acts may occur in a different sequential order and/or one or more of these acts may occur substantially contemporaneously.

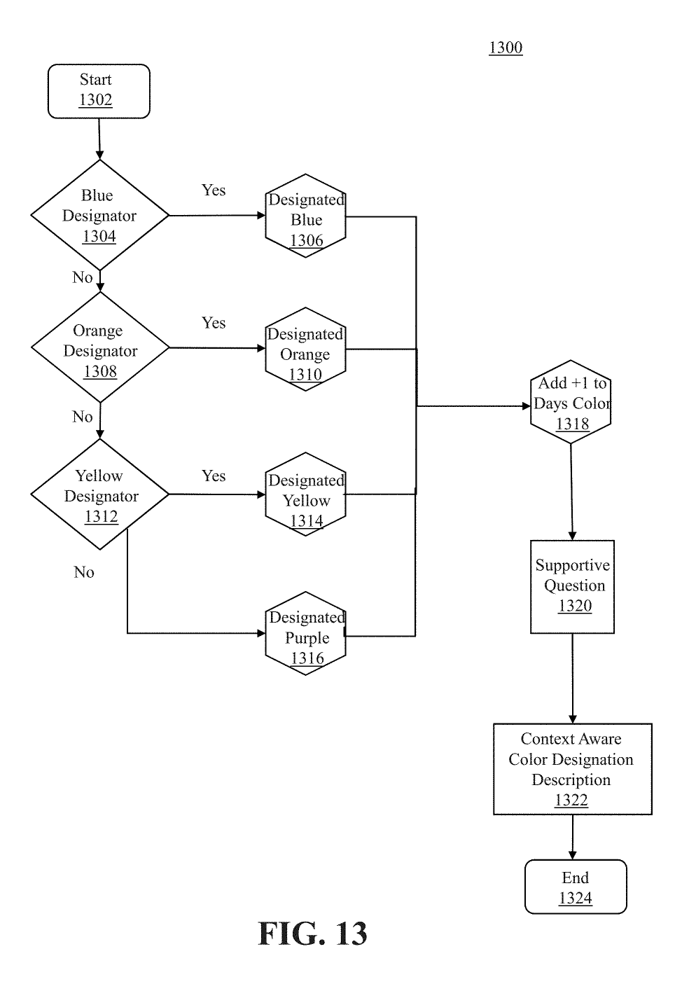

The acts of correlating a patient or caregiver's response to at least one designating query with the severity level of a biomarker (e.g., act 806 in FIG. 8 and act 906 in FIG. 9) may be implemented in a variety of ways. FIGS. 13 through 16 show four representative flowcharts outlining exemplary processes for designating the severity level of a biomarker in accordance with the present teachings. A common feature of these processes is that every time an affirmative response is received to a designating query, the severity level is designated the corresponding color. If a negative response is received, the process proceeds to the next designating query. Although the processes shown in the representative flowcharts in FIGS. 13 through 15 begin by displaying a designating query associated with a blue level of severity, it is to be understood that in alternative embodiments, any color level may be initially queried.

FIG. 13 shows a flowchart of a representative first process 1300 (“Forced Designation”) for designating the severity level of a biomarker in accordance with the present teachings. The process begins at block 1302. At decision block 1304 in FIG. 13, a blue designating query is presented to the user. If the response to this query is “yes,” the severity level is designated “blue,” as shown at block 1306. If the response to this query is “no,” an orange designating query is presented to the user, as shown at block 1308. If the response to this query is “yes,” the severity level is designated “orange,” as shown at block 1310. If the response to this query is “no,” a yellow designating query is presented to the user, as shown at block 1312. If the response to this query is “yes,” the severity level is designated “yellow,” as shown at block 1314. If the response to this query is “no,” the severity level is designated “purple,” as shown at block 1316. Once the severity level has been designated, the process continues to block 1318 where a timing element is added based on the number of days the patient has been assigned the designated severity level (e.g., for purposes including but not limited to tailoring a bespoke designation description individualized for the patient). At block 1320, a supportive query is presented to the user, and at block 1322, a contextualized designation description is presented to the user. The process concludes at block 1324.

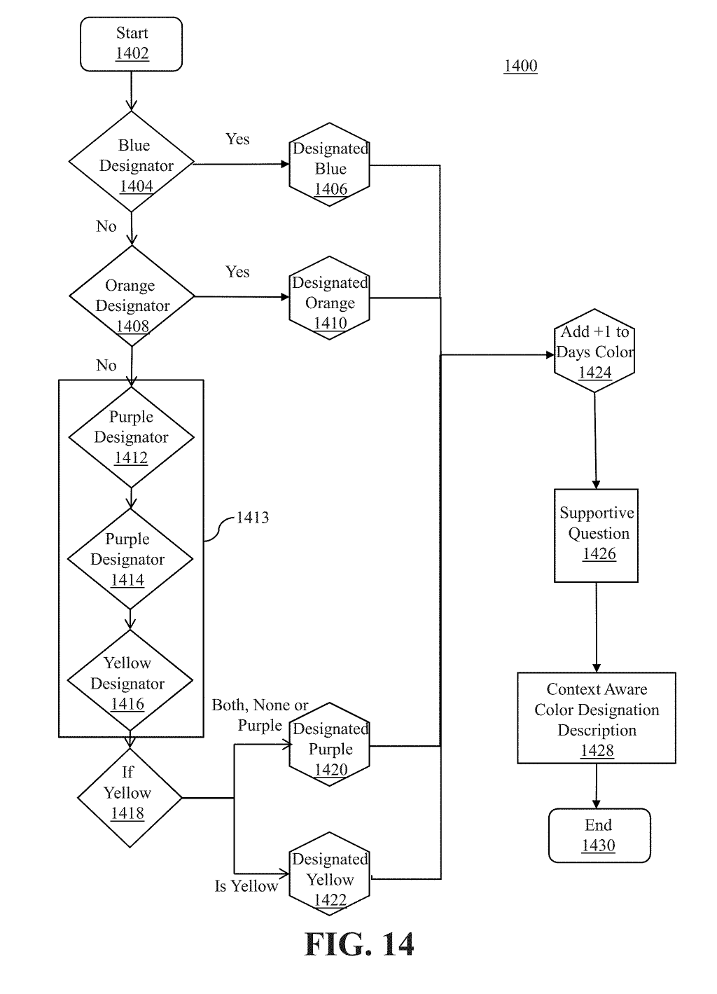

FIG. 14 shows a flowchart of a representative second process 1400 (“Ask All”) for designating the severity level of a biomarker in accordance with the present teachings. In the flowchart of FIG. 14, all of the different designating queries may be presented to the user. Once a determination is made as to which designating query is true, a severity level may then be assigned. The flowchart shown in FIG. 14 may be well adapted for applications in which the patient or caregiver is already in possession of certain medical facts prior to using the tool (e.g., a breast cancer diagnosis has already been received) but would now like support regarding the available choices and/or treatments, as well as information regarding how the diagnostics and/or treatments work. The process in FIG. 14 begins at block 1402. At decision block 1404 in FIG. 14, a blue designating query is presented to the user. If the response to this query is “yes,” the severity level is designated “blue,” as shown at block 1406. If the response to this query is “no,” an orange designating query is presented to the user, as shown at block 1408. If the response to this query is “yes,” the severity level is designated “orange,” as shown at block 1410. If the response to this query is “no,” a first purple designating query 1412 followed by a second purple designating query 1414 followed by a yellow designated query 1416 are presented to the user, as shown inside block 1413. The multiplicity of purple designating queries inside block 1413 is merely illustrative and is meant to convey that in some embodiments, an affirmative response to multiple designating queries may be required before a severity level is conclusively assigned. At decision block 1418 in FIG. 14, a determination is made as to whether the severity level is “yellow.” If the response to the query presented at block 1418 is “both, none or purple,” the severity level is designated “purple,” as shown at block 1420. If the response to the query presented at block 1418 is “yellow,” the severity level is designated “yellow,” as shown at block 1422. Once the severity level has been designated, the process continues to block 1424 where a timing element is added based on the number of days the patient has been assigned the designated severity level (e.g., for purposes including but not limited to tailoring a bespoke designation description individualized for the patient). At block 1426, a supportive query is presented to the user, and at block 1428, a contextualized designation description is presented to the user. The process concludes at block 1430.

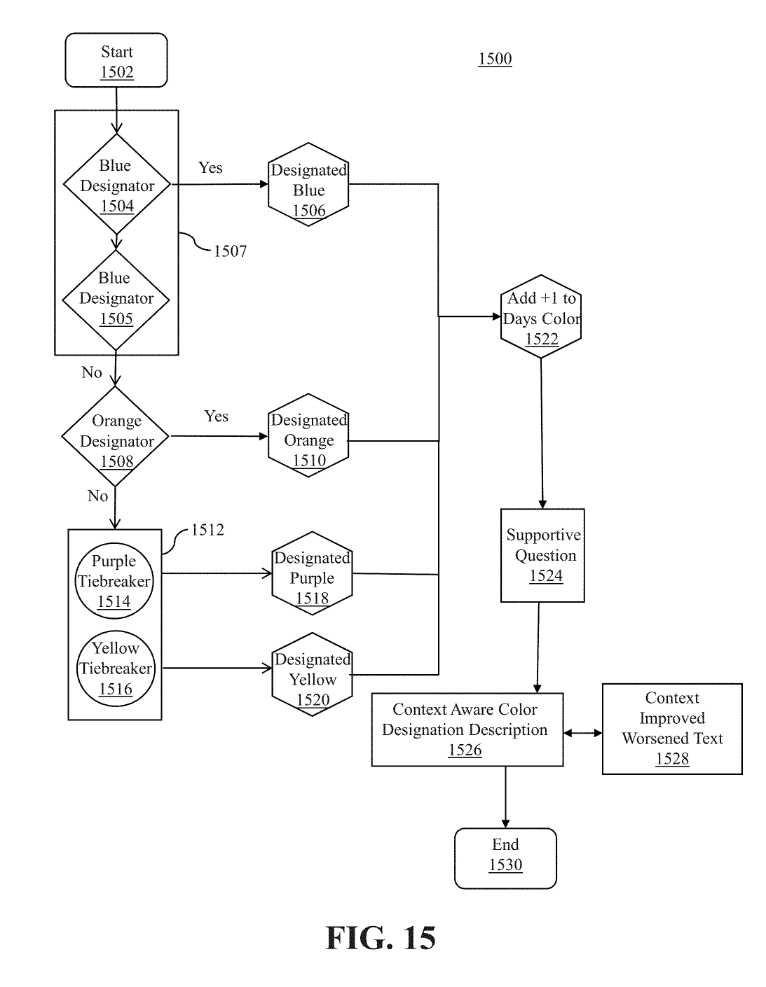

FIG. 15 shows a flowchart of a representative third process 1500 (“Tiebreaker”) for designating the severity level of a biomarker in accordance with the present teachings. In the flowchart of FIG. 15, the designating query that is most likely true is presented first. On the chance that the query is answered negatively, one additional designating query is then presented. If the answer to this second query is once again negative, a tiebreaker is instituted. In the tiebreaker, the user is presented with the two remaining designating queries and allowed to choose which one is applicable rather than having to respond to both queries individually. The flowchart shown in FIG. 15 may be well adapted for neonatal applications involving preemies since the parents of NICU infants may be under emotional stress and, therefore, less willing to respond to a multiplicity of queries. The process in FIG. 15 begins at block 1502. In block 1507 in FIG. 15, a first blue designating query 1504 is presented to the user followed by a second designating query 1505. The multiplicity of blue designating queries shown inside block 1507 is intended as being merely illustrative and is meant to convey that in some embodiments, a negative response to multiple designating queries may be required before a severity level is conclusively ruled out. If the response to the first blue designating query 1504 and/or the second blue designating query 1505 shown inside block 1507 is “yes,” the severity level is designated “blue,” as shown at block 1506. If the response to the first blue designating query 1504 and the second blue designating query 1505 shown inside block 1507 is “no,” an orange designating query is presented to the user, as shown at block 1508. If the response to this query is “yes,” the severity level is designated “orange,” as shown at block 1510. If the response to this query is “no,” the process enters a tiebreaker, as shown at block 1512. In the tiebreaker 1512, a purple designating query 1514 and a yellow designating query 1516 are presented to the user who may choose which query is applicable to the patient. If the response to the purple designating query is “yes,” the severity level is designated “purple,” as shown at block 1518. If the response to the yellow designating query is “yes,” the severity level is designated “yellow,” as shown at block 1520. Once the severity level has been designated, the process continues to block 1522 where a timing element is added based on the number of days the patient has been assigned the designated severity level (e.g., for purposes including but not limited to tailoring a bespoke designation description individualized for the patient). At block 1524, a supportive query is presented to the user. At block 1526, a contextualized designation description is presented to the user. This bespoke designation description may be further individualized based on whether the designated severity level is the result of an improvement or worsening in the patient's condition. The process concludes at block 1530. It should be noted that in FIG. 15, the tiebreaker 1512 is shown as being between purple 1514 and yellow 1516 for purposes of illustration. However, the tiebreaker is relative to the two colors between which a determination is to be made. Thus, if the tiebreaker were between two different colors, the nature of the designating queries needed to differentiate between the two colors may be different from what is needed to differentiate between purple and yellow as shown in FIG. 15.

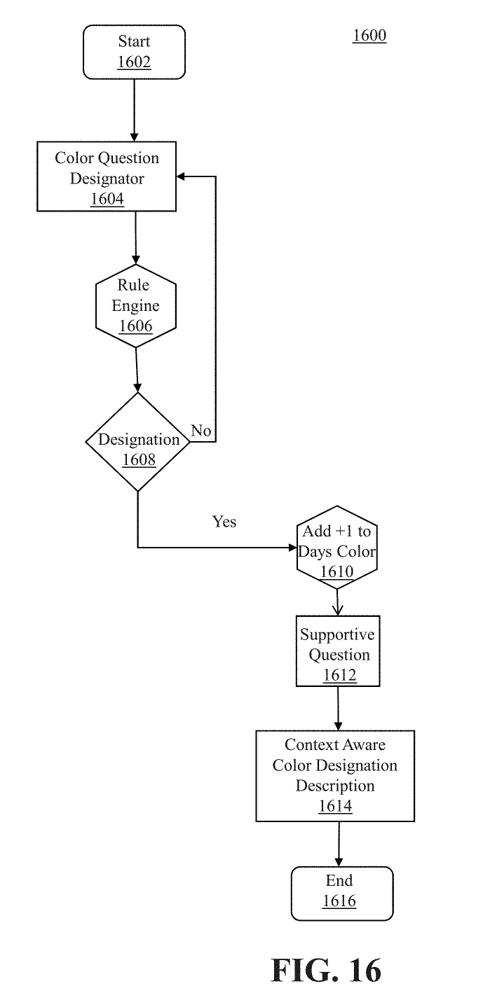

FIG. 16 shows a flowchart of a representative fourth process 1600 (“Question Set”) for designating the severity level of a biomarker in accordance with the present teachings. In the flowchart of FIG. 16, both designating queries and supportive queries for a particular color are presented to the user in sequence and are not separated out in contrast to FIGS. 13, 14, and 15. Although affirmative responses to one or more specific queries are still the basis for designating the severity level of a given biomarker, a rule engine is used to determine whether the appropriate queries have been answered affirmatively before the severity level is designated. The process begins at block 1602. At decision block 1604 in FIG. 16, one or more queries (e.g., a combination of one or more designating queries and one or more supportive queries) are presented to a user. The responses to these queries are fed to a rule engine 1606 which determines whether the appropriate queries and/or the appropriate combination of queries have been answered affirmatively. If the rule engine 1606 determines that the answer is “yes,” the process proceeds to block 1608 where a designated severity level is assigned. If the rule engine 1606 determines that the answer is “no,” the process recycles to block 1604 and additional queries associated with a different color are then presented to the user for consideration. Once the severity level has been designated, the process continues to block 1610 where a timing element is added based on the number of days the patient has been assigned the designated severity level (e.g., for purposes including but not limited to tailoring a bespoke designation description individualized for the patient). At block 1612, a supportive query is presented to the user, and at block 1614, a contextualized designation description is presented to the user. The process concludes at block 1616.

It is to be understood that the relative ordering of some acts shown in the flowcharts of FIGS. 13 through 16 are meant to be merely representative rather than limiting, and that alternative sequences may be followed. Moreover, it is likewise to be understood that additional, different, or fewer acts may be provided, and that in alternative embodiments, two or more of the acts may occur sequentially, substantially contemporaneously, and/or in alternative orders.

In some embodiments, as described above, the present teachings provide methods for guiding a patient or a caregiver thereof along a medical trajectory associated with a medical problem of the patient. In other embodiments, as further described below, the present teachings also provide systems for guiding a patient or a caregiver thereof along a medical trajectory associated with a medical problem of the patient.

By way of example, a first system for guiding a patient or a caregiver thereof along a medical trajectory associated with a medical problem of the patient in accordance with the present teachings includes a processor coupled to a non-transitory memory, wherein the processor is operative to execute computer program instructions to cause the processor to: (a) display at least one designating query to the patient and/or the caregiver, the at least one designating query designed to ascertain a severity level of a first biomarker associated with the medical problem; (b) receive a response from the patient and/or the caregiver to the at least one designating query; (c) correlate the response to the at least one designating query with a severity level of the first biomarker; and (d) display a designated severity level of the first biomarker in a graphical format.

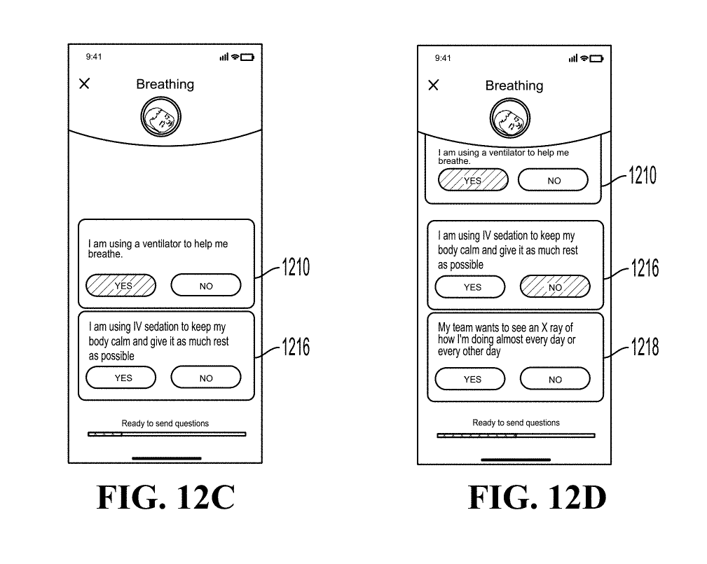

Further aspects of the present teachings will now be described in reference to the drawings. FIG. 10 shows a block diagram of a representative second system 1000 for guiding a patient or a caregiver thereof along a medical trajectory associated with a medical problem of the patient in accordance with the present teachings. In some embodiments, as shown in FIG. 10, a system 1000 in accordance with the present teachings is implemented as part of a GPU in a computer system. In other embodiments, the system 1000 may be implemented as part of a CPU in a computer system.

In some embodiments, as shown in FIG. 10, a system 1000 for guiding a patient or a caregiver thereof along a medical trajectory associated with a medical problem of the patient in accordance with the present teachings is implemented as part of a guidance module in a computer system. As shown in FIG. 10, the system 1000 includes: a processor 1002; a non-transitory memory 1004 coupled with the processor 1002; first logic 1006 stored in the non-transitory memory 1004 and executable by the processor 1002 to cause the processor 1002 to display at least one designating query to the patient and/or the caregiver, the at least one designating query designed to ascertain a severity level of a first biomarker associated with the medical problem; second logic 1008 stored in the non-transitory memory 1004 and executable by the processor 1002 to cause the processor 1002 to receive a response from the patient and/or the caregiver to the at least one designating query; third logic 1010 stored in the non-transitory memory 1004 and executable by the processor 1002 to cause the processor 1002 to correlate the response to the at least one designating query with a severity level of the first biomarker; and fourth logic 1012 stored in the non-transitory memory 1004 and executable by the processor 1002 to cause the processor 1002 to display a designated severity level of the first biomarker in a graphical format.

In some embodiments, the apparatus 1000 may further include one or more of the following: fifth logic 1014 stored in the non-transitory memory 1004 and executable by the processor 1002 to cause the apparatus 1000 to receive a response from the patient and/or the caregiver to the at least one supportive query; sixth logic 1016 stored in the non-transitory memory 1004 and executable by the processor 1002 to cause the apparatus 1000 to determine a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof; seventh logic 1018 stored in the non-transitory memory 1004 and executable by the processor 1002 to cause the apparatus 1000 to correlate the determined parameter and the designated severity level of the first biomarker with a bespoke designation description relevant to the first biomarker; eighth logic 1020 stored in the non-transitory memory 1004 and executable by the processor 1002 to cause the apparatus 1000 to correlate the determined parameter and the response to the at least one supportive query with bespoke supportive information relevant to the first biomarker; ninth logic 1022 stored in the non-transitory memory 1004 and executable by the processor 1002 to cause the apparatus 1000 to display a designated severity level of the first biomarker in a graphical format; tenth logic 1024 stored in the non-transitory memory 1004 and executable by the processor 1002 to cause the apparatus 1000 to display the bespoke designation description relevant to the first biomarker in a graphical format; eleventh logic 1026 stored in the non-transitory memory 1004 and executable by the processor 1002 to cause the apparatus 1000 to display the bespoke supportive information relevant to the first biomarker in a graphical format; twelfth logic 1028 stored in the non-transitory memory 1004 and executable by the processor 1002 to cause the apparatus 1000 to display at least one additional designating query to the patient and/or the caregiver, the at least one additional designating query designed to ascertain a severity level of at least one additional biomarker associated with the medical problem; thirteenth logic 1030 stored in the non-transitory memory 1004 and executable by the processor 1002 to cause the apparatus 1000 to receive a response from the patient and/or the caregiver to the at least one additional designating query; fourteenth logic 1032 stored in the non-transitory memory 1004 and executable by the processor 1002 to cause the apparatus 1000 to correlate the response to the at least one additional designating query with a severity level of the at least one additional biomarker; fifteenth logic 1034 stored in the non-transitory memory 1004 and executable by the processor 1002 to cause the apparatus 1000 to display at least one additional supportive query to the patient and/or the caregiver, the at least one additional supportive query designed to elicit information relevant to the at least one additional biomarker without influencing a designation of the severity level thereof; sixteenth logic 1036 stored in the non-transitory memory 1004 and executable by the processor 1002 to cause the apparatus 1000 to receive a response from the patient and/or the caregiver to the at least one additional supportive query; seventeenth logic 1038 stored in the non-transitory memory 1004 and executable by the processor 1002 to cause the apparatus 1000 to determine a parameter selected from the group consisting of (v) a duration of time for which the designated severity level of the at least one additional biomarker has been assigned to the patient, (vi) a number of times that the patient has previously been assigned the designated severity level of the at least one additional biomarker, and (vii) a combination thereof; eighteenth logic 1040 stored in the non-transitory memory 1004 and executable by the processor 1002 to cause the apparatus 1000 to correlate the determined parameter and the designated severity level of the at least one additional biomarker with a bespoke designation description relevant to the at least one additional biomarker; nineteenth logic 1042 stored in the non-transitory memory 1004 and executable by the processor 1002 to cause the apparatus 1000 to correlate the determined parameter and the response to the at least one additional supportive query with bespoke supportive information relevant to the at least one additional biomarker; twentieth logic 1044 stored in the non-transitory memory 1004 and executable by the processor 1002 to cause the apparatus 1000 to display a designated severity level of the at least one additional biomarker in a graphical format; twenty-first logic 1046 stored in the non-transitory memory 1004 and executable by the processor 1002 to cause the apparatus 1000 to display the bespoke designation description relevant to the at least one additional biomarker in a graphical format; twenty-second logic 1048 stored in the non-transitory memory 1004 and executable by the processor 1002 to cause the apparatus 1000 to display the bespoke supportive information relevant to the at least one additional biomarker in a graphical format; and/or twenty-third logic 1050 stored in the non-transitory memory 1004 and executable by the processor 1002 to cause the apparatus 1000 to display the designated severity level of the first biomarker and the designated severity level of the at least one additional biomarker via color coding.

In some embodiments, the system 1000 may be coupled to other modules of a computer system and/or to databases so as to have access to relevant information as needed (e.g., medical records, real-time physiological data, HIPPA-compliant servers, etc.) and initiate appropriate actions.

In some embodiments, the system 1000 is configured as a device selected from the group consisting of mobile phones, personal computers, laptops, notebooks, tablets, portable media players, personal digital assistants, pagers, game consoles, navigation systems, and the like, and combinations thereof. In some embodiments, the system 1000 is configured as a mobile phone and further includes: (a) user interface circuitry and user interface software configured to (i) facilitate user control of at least some functions of the navigation system and/or mobile phone though use of a display and (ii) respond to user inputs; and (b) a display and display circuitry configured to display at least a portion of a user interface of the navigation system and/or mobile phone, the display and the display circuitry configured to facilitate user control of at least some of the functions of the navigation system and/or mobile phone.

A third system for guiding a patient or a caregiver thereof along a medical trajectory associated with a medical problem of the patient includes: means for displaying at least one designating query to the patient and/or the caregiver, the at least one designating query designed to ascertain a severity level of a first biomarker associated with the medical problem; means for receiving a response from the patient and/or the caregiver to the at least one designating query; means for correlating the response to the at least one designating query with a severity level of the first biomarker; and means for displaying a designated severity level of the first biomarker in a graphical format.

A non-transitory computer-readable storage medium in accordance with the present teachings has stored therein data representing instructions executable by a programmed processor for guiding a patient or a caregiver thereof along a medical trajectory associated with a medical problem of the patient. The storage medium includes instructions for: (a) displaying at least one designating query to the patient and/or the caregiver, the at least one designating query designed to ascertain a severity level of a first biomarker associated with the medical problem; (b) receiving a response from the patient and/or the caregiver to the at least one designating query; (c) correlating the response to the at least one designating query with a severity level of the first biomarker; and (d) displaying a designated severity level of the first biomarker in a graphical format.

One or more modules or logic described herein may be implemented using, among other things, a tangible computer-readable medium comprising computer-executable instructions (e.g., executable software code). Alternatively, modules may be implemented as software code, firmware code, hardware, and/or a combination of the aforementioned. For example the modules may be embodied as part of a HIPPA-compliant server.

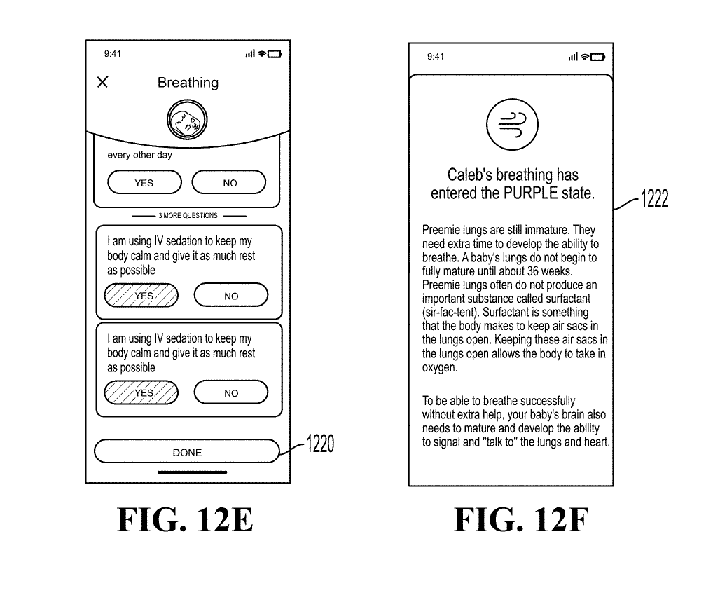

FIG. 11 depicts an illustrative embodiment of a general computer system 1100. The computer system 1100 can include a set of instructions that can be executed to cause the computer system 1100 to perform any one or more of the methods or computer based functions disclosed herein. The computer system 1100 may operate as a standalone device or may be connected (e.g., using a network) to other computer systems or peripheral devices. Any of the components discussed above, such as the processor, may be a computer system 1100 or a component in the computer system 1100.

In a networked deployment, the computer system 1100 may operate in the capacity of a server or as a client user computer in a client-server user network environment, or as a peer computer system in a peer-to-peer (or distributed) network environment. The computer system 1100 may also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In some embodiments, the computer system 1100 may be implemented using electronic devices that provide voice, video or data communication. Further, while a single computer system 1100 is illustrated, the term “system” shall also be taken to include any collection of systems or sub-systems that individually or jointly execute a set, or multiple sets, of instructions to perform one or more computer functions.

As shown in FIG. 11, the computer system 1100 may include a processor 1102, for example a central processing unit (CPU), a graphics-processing unit (GPU), or both. The processor 1102 may be a component in a variety of systems. For example, the processor 1102 may be part of a standard personal computer or a workstation. The processor 1102 may be one or more general processors, digital signal processors, application specific integrated circuits, field programmable gate arrays, servers, networks, digital circuits, analog circuits, combinations thereof, or other now known or later developed devices for analyzing and processing data. The processor 1102 may implement a software program, such as code generated manually (i.e., programmed).

The computer system 1100 may include a memory 1104 that may communicate via a bus 1108. The memory 1104 may be a main memory, a static memory, or a dynamic memory. The memory 1104 may include, but is not limited to, computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In some embodiments, the memory 1104 includes a cache or random access memory for the processor 1102. In alternative embodiments, the memory 1104 is separate from the processor 1102, such as a cache memory of a processor, the system memory, or other memory. The memory 1104 may be an external storage device or database for storing data. Examples include a hard drive, compact disc (CD), digital video disc (DVD), memory card, memory stick, floppy disc, universal serial bus (USB) memory device, or any other device operative to store data. The memory 1104 is operable to store instructions executable by the processor 1102. The functions, acts or tasks illustrated in the figures or described herein may be performed by the programmed processor 1102 executing the instructions 1112 stored in the memory 1104. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown in FIG. 11, the computer system 1100 may further include a display unit 1114, such a touch screen display (e.g., the touch screen of a mobile phone or tablet), a liquid crystal display (LCD), an organic light emitting diode (OLED), a flat panel display, a solid state display, a cathode ray tube (CRT), a projector, a printer or other now known or later developed display device for outputting determined information. The display 1114 may act as an interface for the user to see the functioning of the processor 1102, or specifically as an interface with the software stored in the memory 1104 or in the drive unit 1106. A graphical format in accordance with the present teachings may be output to the user on the display unit 1114. For example, a color-coded image representing the severity level of one or a plurality of biomarkers associated with a patient's medical problem may be included in the image.

Additionally, as shown in FIG. 11, the computer system 1100 may include an input device 1116 configured to allow a user to interact with any of the components of system 1100. The input device 1116 may be a number pad, a keyboard, or a cursor control device, such as a mouse, or a joystick, touch screen display, remote control or any other device operative to interact with the system 1100.

In some embodiments, as shown in FIG. 11, the computer system 1100 may also include a disk or optical drive unit 1106. The disk drive unit 1106 may include a computer-readable medium 1110 in which one or more sets of instructions 1112 (e.g., software) may be embedded. Further, the instructions 1112 may embody one or more of the methods or logic as described herein. In some embodiments, the instructions 1112 may reside completely, or at least partially, within the memory 1104 and/or within the processor 1102 during execution by the computer system 1100. The memory 1104 and the processor 1102 also may include computer-readable media as described above.

The present teachings contemplate a computer-readable medium that includes instructions 1112 or receives and executes instructions 1112 responsive to a propagated signal, so that a device connected to a network 1120 may communicate voice, video, audio, images or any other data over the network 1120. Further, the instructions 1112 may be transmitted or received over the network 1120 via a communication interface 1118. The communication interface 1118 may be a part of the processor 1102 or may be a separate component. The communication interface 1118 may be created in software or may be a physical connection in hardware. The communication interface 1118 is configured to connect with a network 1120, external media, the display 1114, or any other components in system 1100, or combinations thereof. The connection with the network 1120 may be a physical connection, such as a wired Ethernet connection or may be established wirelessly as discussed below. Likewise, the additional connections with other components of the system 1100 may be physical connections or may be established wirelessly.

The network 1120 may include wired networks, wireless networks, or combinations thereof. The wireless network may be a cellular telephone network, an 802.11, 802.16, 802.20, or WiMax network. Further, the network 1120 may be a public network, such as the Internet, a private network, such as an intranet, or combinations thereof, and may utilize a variety of networking protocols now available or later developed including, but not limited to TCP/IP based networking protocols.

Embodiments of the subject matter and the functional operations described in this specification may be implemented in digital electronic circuitry, or in computer software, firmware, or hardware, including the structures disclosed in this specification and their structural equivalents, or in combinations of one or more of them. Embodiments of subject matter described in this specification may be implemented as one or more computer program products, for example, one or more modules of computer program instructions encoded on a computer-readable medium for execution by, or to control the operation of, data processing apparatus. While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein. The computer-readable medium may be a machine-readable storage device, a machine-readable storage substrate, a memory device, or a combination of one or more of them. The term “data processing apparatus” encompasses all apparatuses, devices, and machines for processing data, including but not limited to, by way of example, a programmable processor, a computer, or multiple processors or computers. The apparatus may include, in addition to hardware, code that creates an execution environment for the computer program in question (e.g., code that constitutes processor firmware, a protocol stack, a database management system, an operating system, or a combination thereof).

In some embodiments, the computer-readable medium may include a solid-state memory such as a memory card or other package that houses one or more non-volatile read-only memories. Further, the computer-readable medium may be a random access memory or other volatile re-writable memory. Additionally, the computer-readable medium may include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the present teachings are considered to include any one or more of a computer-readable medium or a distribution medium and other equivalents and successor media, in which data or instructions may be stored.

In some embodiments, dedicated hardware implementations, such as application specific integrated circuits, programmable logic arrays and other hardware devices, may be constructed to implement one or more of the methods described herein. Applications that may include the apparatus and systems of various embodiments may broadly include a variety of electronic and computer systems. One or more embodiments described herein may implement functions using two or more specific interconnected hardware modules or devices with related control and data signals that may be communicated between and through the modules, or as portions of an application-specific integrated circuit. Accordingly, the present system encompasses software, firmware, and hardware implementations.

In some embodiments, the methods described herein may be implemented by software programs executable by a computer system. Further, in some embodiments, implementations may include distributed processing, component/object distributed processing, and parallel processing. Alternatively, virtual computer system processing may be constructed to implement one or more of the methods or functionality as described herein.

Although the present teachings describe components and functions that may be implemented in particular embodiments with reference to particular standards and protocols, the present invention is not limited to such standards and protocols. For example, standards for Internet and other packet switched network transmission (e.g., TCP/IP, UDP/IP, HTML, HTTP, HTTPS) represent examples of the state of the art. Such standards are periodically superseded by faster or more efficient equivalents having essentially the same functions. Accordingly, replacement standards and protocols having the same or similar functions as those disclosed herein are considered equivalents thereof.

A computer program (also known as a program, software, software application, script, or code) may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described herein may be performed by one or more programmable processors executing one or more computer programs to perform functions by operating on input data and generating output. The processes and logic flows may also be performed by, and apparatus may also be implemented as, special purpose logic circuitry, for example, an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The main elements of a computer are a processor for performing instructions and one or more memory devices for storing instructions and data. Generally, a computer will also include, or be operatively coupled to receive data from or transfer data to, or both, one or more mass storage devices for storing data, for example, magnetic, magneto optical disks, or optical disks. However, a computer need not have such devices. Moreover, a computer may be embedded in another device, for example, a mobile telephone, a personal digital assistant (PDA), a mobile audio player, a Global Positioning System (GPS) receiver, to name just a few. Computer-readable media suitable for storing computer program instructions and data include all forms of non volatile memory, media and memory devices, including but not limited to, by way of example, semiconductor memory devices (e.g., EPROM, EEPROM, and flash memory devices); magnetic disks (e.g., internal hard disks or removable disks); magneto optical disks; and CD ROM and DVD-ROM disks. The processor and the memory may be supplemented by, or incorporated in, special purpose logic circuitry.

To provide for interaction with a user, some embodiments of subject matter described herein may be implemented on a device having a display, for example a CRT (cathode ray tube) or LCD (liquid crystal display) monitor, for displaying information to the user and a keyboard and a pointing device, for example a mouse or a trackball, by which the user may provide input to the computer. Other kinds of devices may be used to provide for interaction with a user as well. By way of example, feedback provided to the user may be any form of sensory feedback (e.g., visual feedback, auditory feedback, or tactile feedback); and input from the user may be received in any form, including but not limited to acoustic, speech, or tactile input.

Embodiments of subject matter described herein may be implemented in a computing system that includes a back-end component, for example, as a data server, or that includes a middleware component, for example, an application server, or that includes a front end component, for example, a client computer having a graphical user interface or a Web browser through which a user may interact with an implementation of the subject matter described in this specification, or any combination of one or more such back end, middleware, or front end components. The components of the system may be interconnected by any form or medium of digital data communication, for example, a communication network. Examples of communication networks include but are not limited to a local area network (LAN) and a wide area network (WAN), for example, the Internet.

The computing system may include clients and servers. A client and server are generally remote from each other and typically interact through a communication network. The relationship of client and server arises by virtue of computer programs running on the respective computers and having a client-server relationship to each other.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

While this specification contains many specifics, these should not be construed as limitations on the scope of the invention or of what may be claimed, but rather as descriptions of features specific to particular embodiments. Certain features that are described in this specification in the context of separate embodiments may also be implemented in combination in a single embodiment. Conversely, various features that are described in the context of a single embodiment may also be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings and described herein in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 CFR § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding claim—whether independent or dependent—and that such new combinations are to be understood as forming a part of the present specification.

The foregoing detailed description and the accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

1. A computer-implemented method for guiding a patient or a caregiver thereof along a medical trajectory associated with a medical problem of the patient, the method comprising:
displaying, by a processor, at least one designating query to the patient and/or the caregiver, the at least one designating query designed to ascertain a severity level of a first biomarker associated with the medical problem;
receiving a response from the patient and/or the caregiver to the at least one designating query;
correlating the response to the at least one designating query with a severity level of the first biomarker; and
displaying a designated severity level of the first biomarker in a graphical format.

2. The invention of claim 1 further comprising:
correlating the designated severity level of the first biomarker with a designation description relevant to the first biomarker; and
displaying the designation description relevant to the first biomarker in a graphical format.

3. The invention of claim 1 further comprising:
determining a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof;
correlating the determined parameter with a bespoke designation description relevant to the first biomarker; and
displaying the bespoke designation description relevant to the first biomarker in a graphical format.

4. The invention of claim 1 wherein the first biomarker is one of a plurality of biomarkers associated with the medical problem.

5. The invention of claim 1 further comprising:
displaying at least one additional designating query to the patient and/or the caregiver, the at least one additional designating query designed to ascertain a severity level of at least one additional biomarker associated with the medical problem;
receiving a response from the patient and/or the caregiver to the at least one additional designating query;
correlating the response to the at least one additional designating query with a severity level of the at least one additional biomarker; and
displaying a designated severity level of the at least one additional biomarker in a graphical format.

6. The invention of claim 5 further comprising:
correlating the designated severity level of the at least one additional biomarker with a designation description relevant to the at least one additional biomarker; and
displaying the designation description relevant to the at least one additional biomarker in a graphical format.

7. The invention of claim 5 further comprising:
determining a parameter selected from the group consisting of (i) a duration of time for which the designated severity level of the at least one additional biomarker has been assigned to the patient, (ii) a locale of the patient's medical treatment, (iii) a number of times that the patient has previously been assigned the designated severity level of the at least one additional biomarker, and (iv) a combination thereof;
correlating the determined parameter with a bespoke designation description relevant to the at least one additional biomarker; and
displaying the bespoke designation description relevant to the at least one additional biomarker in a graphical format.

8. The invention of claim 5 wherein each of the first biomarker and the at least one additional biomarker is assigned a relative importance, wherein the first biomarker is assigned primary importance, and wherein severity levels of the plurality of biomarkers are ascertained sequentially in order of decreasing importance of the corresponding biomarker.

9. The invention of claim 1 further comprising:
displaying at least one supportive query to the patient and/or the caregiver, the at least one supportive query designed to elicit information relevant to the first biomarker without influencing designation of the severity level thereof;
receiving a response from the patient and/or the caregiver to the at least one supportive query;
correlating the response to the at least one supportive query with supportive information relevant to the first biomarker; and
displaying the supportive information relevant to the first biomarker in a graphical format.

10. The invention of claim 1 further comprising:
displaying at least one supportive query to the patient and/or the caregiver, the at least one supportive query designed to elicit information relevant to the first biomarker without influencing designation of the severity level thereof;
receiving a response from the patient and/or the caregiver to the at least one supportive query;
determining a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof;
correlating the determined parameter and the response to the at least one supportive query with bespoke supportive information relevant to the first biomarker; and
displaying the bespoke supportive information relevant to the first biomarker in a graphical format.

11. The invention of claim 9 wherein the first biomarker is one of a plurality of biomarkers associated with the medical problem.

12. The invention of claim 9 further comprising:
displaying at least one additional supportive query to the patient and/or the caregiver, the at least one additional supportive query designed to elicit information relevant to the at least one additional biomarker without influencing a designation of the severity level thereof
receiving a response from the patient and/or the caregiver to the at least one additional supportive query;
correlating the response to the at least one additional supportive query with supportive information relevant to the at least one additional biomarker; and
displaying the supportive information relevant to the at least one additional biomarker in a graphical format.

13. The invention of claim 1 further comprising:
displaying at least one additional supportive query to the patient and/or the caregiver, the at least one additional supportive query designed to elicit information relevant to the at least one additional biomarker without influencing a designation of the severity level thereof
receiving a response from the patient and/or the caregiver to the at least one additional supportive query;
determining a parameter selected from the group consisting of (i) a duration of time for which the designated severity level of the at least one additional biomarker has been assigned to the patient, (ii) a locale of the patient's medical treatment, (iii) a number of times that the patient has previously been assigned the designated severity level of the at least one additional biomarker, and (iv) a combination thereof;
correlating the determined parameter and the response to the at least one additional supportive query with bespoke supportive information relevant to the at least one additional biomarker; and
displaying the bespoke supportive information relevant to the at least one additional biomarker in a graphical format.

14. The invention of claim 1 wherein the designated severity level of the first biomarker is color-coded.

15. The invention of claim 5 wherein the designated severity level of the first biomarker and the designated severity level of the at least one additional biomarker are independently color-coded.

16. The invention of claim 1 wherein the at least one designating query is designed to be answerable by a patient or a caregiver without medical training.

17. The invention of claim 1 wherein the at least one designating query is designed to be answerable by a medical professional.

18. The invention of claim 1 wherein the patient is a premature infant, and wherein the medical problem comprises issues associated with premature birth.

19. The invention of claim 18 wherein the at least one designating query is designed to be answerable by a familial caregiver without medical training.

20. The invention of claim 4 wherein the patient is a premature infant, wherein the medical problem comprises issues associated with premature birth, and wherein the plurality of biomarkers comprises breathing, eating, temperature control, sleeping, and growth.

21. The invention of claim 20 wherein each of the breathing biomarker, the eating biomarker, the temperature control biomarker, the sleeping biomarker, and the growth biomarker is assigned a relative importance, wherein the breathing biomarker is assigned primary importance, and wherein severity levels of the plurality of biomarkers are ascertained sequentially in order of decreasing importance of the corresponding biomarker.

22. The invention of claim 18 wherein the medical trajectory comprises the premature infant's path from birth through hospital discharge.

23. The invention of claim 1 wherein the medical problem is selected from the group consisting of premature birth, cancer, heart disease, stroke, infection, diabetes, influenza, pneumonia, joint replacement, organ transplant, surgery, and combinations thereof.

24. The invention of claim 1 wherein the at least one designating query is answerable by either an affirmative or negative binary response.

25. The invention of claim 1 wherein the at least one designating query is answerable by a non-binary response.

26. A computer-implemented method for guiding a patient or a caregiver thereof along a medical trajectory associated with a medical problem of the patient, the method comprising:
displaying, by a processor, at least one designating query to the patient and/or the caregiver, the at least one designating query being designed to ascertain a severity level of a first biomarker in a plurality of biomarkers associated with the medical problem, wherein each of the plurality of biomarkers has an assigned relative importance, and wherein severity levels of each of the plurality of biomarkers are ascertained sequentially in order of decreasing importance of the corresponding biomarker;
receiving a response from the patient and/or the caregiver to the at least one designating query;
correlating the response to the at least one designating query with a severity level of the first biomarker, wherein the first biomarker has primary importance among the plurality of biomarkers;
displaying at least one supportive query to the patient and/or the caregiver, the at least one supportive query designed to elicit information relevant to the first biomarker without influencing designation of the severity level thereof;
receiving a response from the patient and/or the caregiver to the at least one supportive query;
determining a first parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof;
correlating the determined first parameter and the designated severity level of the first biomarker with a bespoke designation description relevant to the first biomarker;
correlating the determined first parameter and the response to the at least one supportive query with bespoke supportive information relevant to the first biomarker;
displaying a designated severity level of the first biomarker in a graphical format;
displaying the bespoke designation description relevant to the first biomarker in a graphical format; and
displaying the bespoke supportive information relevant to the first biomarker in a graphical format.

27. The invention of claim 26 further comprising:
displaying at least one additional designating query to the patient and/or the caregiver, the at least one additional designating query designed to ascertain a severity level of at least one additional biomarker associated with the medical problem;
receiving a response from the patient and/or the caregiver to the at least one additional designating query;
correlating the response to the at least one additional designating query with a severity level of the at least one additional biomarker;
displaying at least one additional supportive query to the patient and/or the caregiver, the at least one additional supportive query designed to elicit information relevant to the at least one additional biomarker without influencing a designation of the severity level thereof;
receiving a response from the patient and/or the caregiver to the at least one additional supportive query;
determining a second parameter selected from the group consisting of (i) a duration of time for which the designated severity level of the at least one additional biomarker has been assigned to the patient, (ii) a number of times that the patient has previously been assigned the designated severity level of the at least one additional biomarker, and (iii) a combination thereof;
correlating the determined parameter and the designated severity level of the at least one additional biomarker with a bespoke designation description relevant to the at least one additional biomarker;
correlating the determined parameter and the response to the at least one additional supportive query with bespoke supportive information relevant to the at least one additional biomarker;
displaying a designated severity level of the at least one additional biomarker in a graphical format;
displaying the bespoke designation description relevant to the at least one additional biomarker in a graphical format; and
displaying the bespoke supportive information relevant to the at least one additional biomarker in a graphical format.

28. A computer-implemented method for guiding a caregiver of a premature infant along a medical trajectory associated with the infant's organ maturation, the method comprising:
displaying, by a processor, at least one designating query to the caregiver, the at least one designating query designed to ascertain a severity level of a breathing biomarker;
receiving a response from the caregiver to the at least one designating query;
correlating the response to the at least one designating query with a severity level of the breathing biomarker, wherein the breathing biomarker is assigned primary importance among a plurality of biomarkers associated with organ maturation of the infant;
displaying at least one supportive query to the caregiver, the at least one supportive query designed to elicit information relevant to the breathing biomarker without influencing designation of the severity level thereof;
receiving a response from the caregiver to the at least one supportive query;
displaying at least one additional designating query to the caregiver, the at least one additional designating query designed to ascertain a severity level of at least one additional biomarker associated with the organ maturation, the at least one additional biomarker selected from the group consisting of eating, temperature control, sleeping, growth, and a combination thereof;
receiving a response from the caregiver to the at least one additional designating query;
correlating the response to the at least one additional designating query with a severity level of the at least one additional biomarker;
displaying at least one additional supportive query to the caregiver, the at least one additional supportive query designed to elicit information relevant to the at least one additional biomarker without influencing a designation of the severity level thereof;
receiving a response from the caregiver to the at least one additional supportive query;
determining a parameter selected from the group consisting of (i) a duration of time for which the designated severity level of the breathing biomarker has been assigned to the patient, (ii) a locale of the infant's medical treatment, (iii) a number of times that the infant has previously been assigned the designated severity level of the breathing biomarker, (iv) a duration of time for which the designated severity level of the at least one additional biomarker has been assigned to the infant, (v) a number of times that the infant has previously been assigned the designated severity level of the at least one additional biomarker, and (vi) a combination thereof;
correlating the determined parameter and the designated severity level of the breathing biomarker with a bespoke designation description relevant to the breathing biomarker;
correlating the determined parameter and the designated severity level of the at least one additional biomarker with a bespoke designation description relevant to the at least one additional biomarker;
correlating the determined parameter and the response to the at least one supportive query with bespoke supportive information relevant to the breathing biomarker;
correlating the determined parameter and the response to the at least one additional supportive query with bespoke supportive information relevant to the at least one additional biomarker;
displaying a designated severity level of the breathing biomarker in a graphical format;
displaying a designated severity level of the at least one additional biomarker in a graphical format;
displaying the bespoke designation description relevant to the breathing biomarker in a graphical format;
displaying the bespoke supportive information relevant to the breathing biomarker in a graphical format;
displaying the bespoke designation description relevant to the at least one additional biomarker in a graphical format; and
displaying the bespoke supportive information relevant to the at least one additional biomarker in a graphical format.

29. The invention of claim 28 wherein the designated severity level of the breathing biomarker and the designated severity level of the at least one additional biomarker are independently color-coded, and wherein each of the designation description relevant to the breathing biomarker, the supportive information relevant to the breathing biomarker, the designation description relevant to the at least one additional biomarker, and the supportive information relevant to the at least one additional biomarker comprises text.

30. The invention of claim 29 wherein one or more of the at least one designating query, the at least one supportive query, the at least one additional designating query, and the at least one additional supportive query is answerable by either an affirmative or a negative binary response.

31. The invention of claim 29 wherein each of the at least one designating query, the at least one supportive query, the at least one additional designating query, and the at least one additional supportive query is answerable by either an affirmative or a negative binary response.

32. A system for guiding a patient or a caregiver thereof along a medical trajectory associated with a medical problem of the patient, the system comprising:
a processor coupled to a non-transitory memory, wherein the processor is operative to execute computer program instructions to cause the processor to:
(a) display at least one designating query to the patient and/or the caregiver, the at least one designating query designed to ascertain a severity level of a first biomarker associated with the medical problem;
(b) receive a response from the patient and/or the caregiver to the at least one designating query;
(c) correlate the response to the at least one designating query with a severity level of the first biomarker; and
(d) display a designated severity level of the first biomarker in a graphical format.

33. A system for guiding a patient or a caregiver thereof along a medical trajectory associated with a medical problem of the patient, the system comprising:
a processor;
a non-transitory memory coupled with the processor;
(a) first logic stored in the non-transitory memory and executable by the processor to cause the processor to display at least one designating query to the patient and/or the caregiver, the at least one designating query designed to ascertain a severity level of a first biomarker associated with the medical problem;
(b) second logic stored in the non-transitory memory and executable by the processor to cause the processor to receive a response from the patient and/or the caregiver to the at least one designating query;
(c) third logic stored in the non-transitory memory and executable by the processor to cause the processor to correlate the response to the at least one designating query with a severity level of the first biomarker; and
(d) fourth logic stored in the non-transitory memory and executable by the processor to cause the processor to display a designated severity level of the first biomarker in a graphical format.

34. The invention of claim 33 further comprising:
(e) fifth logic stored in the non-transitory memory and executable by the processor to cause the processor to correlate the designated severity level of the first biomarker with a designation description relevant to the first biomarker;
(f) sixth logic stored in the non-transitory memory and executable by the processor to cause the processor to display the designation description relevant to the first biomarker in a graphical format.

35. The invention of claim 33 further comprising:
(e) fifth logic stored in the non-transitory memory and executable by the processor to cause the processor to determine a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof;
(f) sixth logic stored in the non-transitory memory and executable by the processor to cause the processor to correlate the determined parameter with a bespoke designation description relevant to the first biomarker; and
(g) seventh logic stored in the non-transitory memory and executable by the processor to cause the processor to display the bespoke designation description relevant to the first biomarker in a graphical format.

36. The invention of claim 33 further comprising:
(e) fifth logic stored in the non-transitory memory and executable by the processor to cause the processor to display at least one additional designating query to the patient and/or the caregiver, the at least one additional designating query designed to ascertain a severity level of at least one additional biomarker associated with the medical problem;
(f) sixth logic stored in the non-transitory memory and executable by the processor to cause the processor to receive a response from the patient and/or the caregiver to the at least one additional designating query;
(g) seventh logic stored in the non-transitory memory and executable by the processor to cause the processor to correlate the response to the at least one additional designating query with a severity level of the at least one additional biomarker; and
(h) eighth logic stored in the non-transitory memory and executable by the processor to cause the processor to display a designated severity level of the at least one additional biomarker in a graphical format.

37. The invention of claim 36 further comprising:
(i) ninth logic stored in the non-transitory memory and executable by the processor to cause the processor to correlate the designated severity level of the at least one additional biomarker with a designation description relevant to the at least one additional biomarker; and
(j) tenth logic stored in the non-transitory memory and executable by the processor to cause the processor to display the designation description relevant to the at least one additional biomarker in a graphical format.

38. The invention of claim 36 further comprising:
(i) ninth logic stored in the non-transitory memory and executable by the processor to cause the processor to determine a parameter selected from the group consisting of (i) a duration of time for which the designated severity level of the at least one additional biomarker has been assigned to the patient, (ii) a locale of the patient's medical treatment, (iii) a number of times that the patient has previously been assigned the designated severity level of the at least one additional biomarker, and (iv) a combination thereof;
(j) tenth logic stored in the non-transitory memory and executable by the processor to cause the processor to correlate the determined parameter with a bespoke designation description relevant to the at least one additional biomarker; and
(k) eleventh logic stored in the non-transitory memory and executable by the processor to cause the processor to display the bespoke designation description relevant to the at least one additional biomarker in a graphical format.

39. The invention of claim 36 further comprising:
(i) ninth logic stored in the non-transitory memory and executable by the processor to cause the processor to assign a relative importance to each of the first biomarker and the at least one additional biomarker, wherein the first biomarker is assigned primary importance; and
(j) tenth logic stored in the non-transitory memory and executable by the processor to cause the processor to ascertain severity levels of the plurality of biomarkers are sequentially in order of decreasing importance of the corresponding biomarker.

40. The invention of claim 33 further comprising:
(e) fifth logic stored in the non-transitory memory and executable by the processor to cause the processor to display at least one supportive query to the patient and/or the caregiver, the at least one supportive query designed to elicit information relevant to the first biomarker without influencing designation of the severity level thereof;
(f) sixth logic stored in the non-transitory memory and executable by the processor to cause the processor to receive a response from the patient and/or the caregiver to the at least one supportive query;
(g) seventh logic stored in the non-transitory memory and executable by the processor to cause the processor to correlate the response to the at least one supportive query with supportive information relevant to the first biomarker; and
(h) eighth logic stored in the non-transitory memory and executable by the processor to cause the processor to display the supportive information relevant to the first biomarker in a graphical format.

41. The invention of claim 33 further comprising:
(e) fifth logic stored in the non-transitory memory and executable by the processor to cause the processor to display at least one supportive query to the patient and/or the caregiver, the at least one supportive query designed to elicit information relevant to the first biomarker without influencing designation of the severity level thereof;
(f) sixth logic stored in the non-transitory memory and executable by the processor to cause the processor to receive a response from the patient and/or the caregiver to the at least one supportive query;
(g) seventh logic stored in the non-transitory memory and executable by the processor to cause the processor to determine a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof;
(h) eighth logic stored in the non-transitory memory and executable by the processor to cause the processor to correlate the determined parameter with bespoke supportive information relevant to the first biomarker; and
(i) ninth logic stored in the non-transitory memory and executable by the processor to cause the processor to display the bespoke supportive information relevant to the first biomarker in a graphical format.

42. The invention of claim 40 further comprising:
(i) ninth logic stored in the non-transitory memory and executable by the processor to cause the processor to display at least one additional supportive query to the patient and/or the caregiver, the at least one additional supportive query designed to elicit information relevant to the at least one additional biomarker without influencing a designation of the severity level thereof;
(j) tenth logic stored in the non-transitory memory and executable by the processor to cause the processor to receive a response from the patient and/or the caregiver to the at least one additional supportive query;
(k) eleventh logic stored in the non-transitory memory and executable by the processor to cause the processor to correlate the response to the at least one additional supportive query with supportive information relevant to the at least one additional biomarker; and
(l) twelfth logic stored in the non-transitory memory and executable by the processor to cause the processor to display the supportive information relevant to the at least one additional biomarker in a graphical format.

43. The invention of claim 33 further comprising:
(e) fifth logic stored in the non-transitory memory and executable by the processor to cause the processor to display at least one additional supportive query to the patient and/or the caregiver, the at least one additional supportive query designed to elicit information relevant to the at least one additional biomarker without influencing a designation of the severity level thereof;
(f) sixth logic stored in the non-transitory memory and executable by the processor to cause the processor to receive a response from the patient and/or the caregiver to the at least one additional supportive query;
(g) seventh logic stored in the non-transitory memory and executable by the processor to cause the processor to determine a parameter selected from the group consisting of (i) a duration of time for which the designated severity level of the at least one additional biomarker has been assigned to the patient, (ii) a locale of the patient's medical treatment, (iii) a number of times that the patient has previously been assigned the designated severity level of the at least one additional biomarker, and (iv) a combination thereof;
(h) eighth logic stored in the non-transitory memory and executable by the processor to cause the processor to correlate the determined parameter with bespoke supportive information relevant to the at least one additional biomarker; and
(i) ninth logic stored in the non-transitory memory and executable by the processor to cause the processor to display the bespoke supportive information relevant to the at least one additional biomarker in a graphical format.

44. The invention of claim 33 further comprising:
(e) fifth logic stored in the non-transitory memory and executable by the processor to cause the processor to display the designated severity level of the first biomarker and the designated severity level of the at least one additional biomarker via color coding.

45. The invention of claim 33 wherein the patient is a premature infant, wherein the medical problem comprises issues associated with premature birth, wherein the first biomarker comprises a breathing biomarker, wherein the at least one additional biomarker is selected from the group consisting of eating, temperature control, sleeping, growth, and a combination thereof, and wherein the invention further comprises:
(e) fifth logic stored in the non-transitory memory and executable by the processor to cause the processor to assign a relative importance to each of the breathing biomarker, the eating biomarker, the temperature control biomarker, the sleeping biomarker, and the growth biomarker, wherein the breathing biomarker is assigned primary importance; and
(f) sixth logic stored in the non-transitory memory and executable by the processor to cause the processor to ascertain severity levels of the breathing biomarker, the eating biomarker, the temperature control biomarker, the sleeping biomarker, and the growth biomarker sequentially in order of decreasing importance of the corresponding biomarker.

46. A system for guiding a patient or a caregiver thereof along a medical trajectory associated with a medical problem of the patient, the system comprising:
a processor;
a non-transitory memory coupled with the processor;
(a) first logic stored in the non-transitory memory and executable by the processor to cause the processor to display at least one designating query to the patient and/or the caregiver, the at least one designating query being designed to ascertain a severity level of a first biomarker in a plurality of biomarkers associated with the medical problem, wherein each of the plurality of biomarkers has an assigned relative importance, and wherein severity levels of each of the plurality of biomarkers are ascertained sequentially in order of decreasing importance of the corresponding biomarker;
(b) second logic stored in the non-transitory memory and executable by the processor to cause the processor to receive a response from the patient and/or the caregiver to the at least one designating query;
(c) third logic stored in the non-transitory memory and executable by the processor to cause the processor to correlate the response to the at least one designating query with a severity level of the first biomarker, wherein the first biomarker has primary importance among the plurality of biomarkers;
(d) fourth logic stored in the non-transitory memory and executable by the processor to cause the processor to display at least one supportive query to the patient and/or the caregiver, the at least one supportive query designed to elicit information relevant to the first biomarker without influencing designation of the severity level thereof;
(e) fifth logic stored in the non-transitory memory and executable by the processor to cause the processor to receive a response from the patient and/or the caregiver to the at least one supportive query;
(f) sixth logic stored in the non-transitory memory and executable by the processor to cause the processor to determine a parameter selected from the group consisting of (i) a duration of time for which the designated severity level of the first biomarker has been assigned to the patient, (ii) a locale of the patient's medical treatment, (iii) a number of times that the patient has previously been assigned the designated severity level of the first biomarker, and (iv) a combination thereof;
(g) seventh logic stored in the non-transitory memory and executable by the processor to cause the processor to correlate the determined parameter and the designated severity level of the first biomarker with a bespoke designation description relevant to the first biomarker;
(h) eighth logic stored in the non-transitory memory and executable by the processor to cause the processor to correlate the determined parameter and the response to the at least one supportive query with bespoke supportive information relevant to the first biomarker;
(i) ninth logic stored in the non-transitory memory and executable by the processor to cause the processor to display a designated severity level of the first biomarker in a graphical format;
(j) tenth logic stored in the non-transitory memory and executable by the processor to cause the processor to display the bespoke designation description relevant to the first biomarker in a graphical format; and
(k) eleventh logic stored in the non-transitory memory and executable by the processor to cause the processor to display the bespoke supportive information relevant to the first biomarker in a graphical format.

47. The invention of claim 46 further comprising:
(l) twelfth logic stored in the non-transitory memory and executable by the processor to cause the processor to display at least one additional designating query to the patient and/or the caregiver, the at least one additional designating query designed to ascertain a severity level of at least one additional biomarker associated with the medical problem;
(m) thirteenth logic stored in the non-transitory memory and executable by the processor to cause the processor to receive a response from the patient and/or the caregiver to the at least one additional designating query;
(n) fourteenth logic stored in the non-transitory memory and executable by the processor to cause the processor to correlate the response to the at least one additional designating query with a severity level of the at least one additional biomarker;
(o) fifteenth logic stored in the non-transitory memory and executable by the processor to cause the processor to display at least one additional supportive query to the patient and/or the caregiver, the at least one additional supportive query designed to elicit information relevant to the at least one additional biomarker without influencing a designation of the severity level thereof;
(p) sixteenth logic stored in the non-transitory memory and executable by the processor to cause the processor to receive a response from the patient and/or the caregiver to the at least one additional supportive query;
(q) seventeenth logic stored in the non-transitory memory and executable by the processor to cause the processor to determine a parameter selected from the group consisting of (v) a duration of time for which the designated severity level of the at least one additional biomarker has been assigned to the patient, (vi) a number of times that the patient has previously been assigned the designated severity level of the at least one additional biomarker, and (vii) a combination thereof;
(r) eighteenth logic stored in the non-transitory memory and executable by the processor to cause the processor to correlate the determined parameter and the designated severity level of the at least one additional biomarker with a bespoke designation description relevant to the at least one additional biomarker;
(s) nineteenth logic stored in the non-transitory memory and executable by the processor to cause the processor to correlate the determined parameter and the response to the at least one additional supportive query with bespoke supportive information relevant to the at least one additional biomarker;
(t) twentieth logic stored in the non-transitory memory and executable by the processor to cause the processor to display a designated severity level of the at least one additional biomarker in a graphical format;
(u) twenty-first logic stored in the non-transitory memory and executable by the processor to cause the processor to display the bespoke designation description relevant to the at least one additional biomarker in a graphical format; and
(v) twenty-second logic stored in the non-transitory memory and executable by the processor to cause the processor to display the bespoke supportive information relevant to the at least one additional biomarker in a graphical format.

48. The invention of claim 47 further comprising:
(w) twenty-third logic stored in the non-transitory memory and executable by the processor to cause the processor to display the designated severity level of the first biomarker and the designated severity level of the at least one additional biomarker via color coding.

49. A system for guiding a caregiver of a premature infant along a medical trajectory associated with the infant's organ maturation, the system comprising:
a processor;
a non-transitory memory coupled with the processor;
(a) first logic stored in the non-transitory memory and executable by the processor to cause the processor to display at least one designating query to the caregiver, the at least one designating query being designed to ascertain a severity level of a breathing biomarker;
(b) second logic stored in the non-transitory memory and executable by the processor to cause the processor to receive a response from the caregiver to the at least one designating query;
(c) third logic stored in the non-transitory memory and executable by the processor to cause the processor to correlate the response to the at least one designating query with a severity level of the breathing biomarker, wherein the breathing biomarker is assigned primary importance among a plurality of biomarkers associated with organ maturation of the infant;
(d) fourth logic stored in the non-transitory memory and executable by the processor to cause the processor to display at least one supportive query to the caregiver, the at least one supportive query designed to elicit information relevant to the breathing biomarker without influencing designation of the severity level thereof;
(e) fifth logic stored in the non-transitory memory and executable by the processor to cause the processor to receive a response from the caregiver to the at least one supportive query;
(f) sixth logic stored in the non-transitory memory and executable by the processor to cause the processor to display at least one additional designating query to the caregiver, the at least one additional designating query designed to ascertain a severity level of at least one additional biomarker associated with the organ maturation, the at least one additional biomarker selected from the group consisting of eating, temperature control, sleeping, growth, and a combination thereof;
(g) seventh logic stored in the non-transitory memory and executable by the processor to cause the processor to receive a response from the caregiver to the at least one additional designating query;
(h) eighth logic stored in the non-transitory memory and executable by the processor to cause the processor to correlate the response to the at least one additional designating query with a severity level of the at least one additional biomarker;
(i) ninth logic stored in the non-transitory memory and executable by the processor to cause the processor to display at least one additional supportive query to the caregiver, the at least one additional supportive query designed to elicit information relevant to the at least one additional biomarker without influencing a designation of the severity level thereof;
(j) tenth logic stored in the non-transitory memory and executable by the processor to cause the processor to receive a response from the caregiver to the at least one additional supportive query;
(k) eleventh logic stored in the non-transitory memory and executable by the processor to cause the processor to determine a parameter selected from the group consisting of (i) a duration of time for which the designated severity level of the breathing biomarker has been assigned to the patient, (ii) a locale of the infant's medical treatment, (iii) a number of times that the infant has previously been assigned the designated severity level of the breathing biomarker, (iv) a duration of time for which the designated severity level of the at least one additional biomarker has been assigned to the infant, (v) a number of times that the infant has previously been assigned the designated severity level of the at least one additional biomarker, and (vi) a combination thereof;
(l) twelfth logic stored in the non-transitory memory and executable by the processor to cause the processor to correlate the determined parameter and the designated severity level of the breathing biomarker with a bespoke designation description relevant to the breathing biomarker;
(m) thirteenth logic stored in the non-transitory memory and executable by the processor to cause the processor to correlate the determined parameter and the designated severity level of the at least one additional biomarker with a bespoke designation description relevant to the at least one additional biomarker;
(n) fourteenth logic stored in the non-transitory memory and executable by the processor to cause the processor to correlate the determined parameter and the response to the at least one supportive query with bespoke supportive information relevant to the breathing biomarker;
(o) fifteenth logic stored in the non-transitory memory and executable by the processor to cause the processor to correlate the determined parameter and the response to the at least one additional supportive query with bespoke supportive information relevant to the at least one additional biomarker;
(p) sixteenth logic stored in the non-transitory memory and executable by the processor to cause the processor to display a designated severity level of the breathing biomarker in a graphical format;
(q) seventeenth logic stored in the non-transitory memory and executable by the processor to cause the processor to display a designated severity level of the at least one additional biomarker in a graphical format;
(r) eighteenth logic stored in the non-transitory memory and executable by the processor to cause the processor to display the bespoke designation description relevant to the breathing biomarker in a graphical format;
(s) nineteenth logic stored in the non-transitory memory and executable by the processor to cause the processor to display the bespoke supportive information relevant to the breathing biomarker in a graphical format;
(t) twentieth logic stored in the non-transitory memory and executable by the processor to cause the processor to displaying the bespoke designation description relevant to the at least one additional biomarker in a graphical format; and
(u) twenty-first logic stored in the non-transitory memory and executable by the processor to cause the processor to display the bespoke supportive information relevant to the at least one additional biomarker in a graphical format.

50. The invention of claim 49 further comprising:
(v) twenty-second logic stored in the non-transitory memory and executable by the processor to cause the processor to display the respective designated severity levels of the breathing biomarker, the eating biomarker, the temperature control biomarker, the sleeping biomarker, and the growth biomarker via color coding.

51. A non-transitory computer-readable storage medium having stored therein data representing instructions executable by a programmed processor for guiding a patient or a caregiver thereof along a medical trajectory associated with a medical problem of the patient, the storage medium comprising instructions for:
displaying at least one designating query to the patient and/or the caregiver, the at least one designating query designed to ascertain a severity level of a first biomarker associated with the medical problem;
receiving a response from the patient and/or the caregiver to the at least one designating query;
correlating the response to the at least one designating query with a severity level of the first biomarker; and
displaying a designated severity level of the first biomarker in a graphical format.

52-73. (canceled)
